SEQUENCE LISTING (2 sequences)

SEQ ID NO: 1 - DNA, 20 bp, Artificial Sequence
  Synthetic nucleotide
tgcagttccggtggctgatc

SEQ ID NO: 2 - DNA, 20 bp, Artificial Sequence
  Synthetic nucleotide
gatcagccaccggaactgca

(19) United States
(12) Patent Application Publication
(10) Pub. No.: US 2008/0268546 A1
(43) Pub. Date: Oct. 30, 2008

(54) DETECTION OF DNA HYBRIDIZATION ON SURFACES

(75) Inventors: Nicholas L. Abbott, Madison, WI (US); Jeffrey M. Brake, Madison, WI (US); Pritipal S. Bhinder, San Diego, CA (US)

Correspondence Address: Wisconsin Alumni Research Foundation (WARF), C/O Foley & Lardner LLP, Verex Plaza, 150 East Gillman Street, Madison, WI, 53703-1481, US

(73) Assignee: WISCONSIN ALUMNI RESEARCH FOUNDATION

(21) Appl. No.: 12/032,107

(22) Filed: Feb. 15, 2008

Related U.S. Application Data
(62) Division of application No. 10/262,562, filed on Oct. 1, 2002, now abandoned.
(60) Provisional application No. 60/327,138, filed on Oct. 4, 2001.

(51) Int. Cl. G01N 33/00

(52) U.S. Cl. 436/94

(57) ABSTRACT

A DNA hybridization surface includes a support having a self assembled monolayer on a metallized surface. The self assembled monolayer includes an alkanethiol and a strand of nucleic acids comprising a functional group that binds to the metallized surface. A method for detecting DNA hybridization in a sample includes (a) incubating a DNA hybridization surface with an aqueous sample that includes a fragment of DNA to produce an incubated DNA hybridization surface; (b) rinsing the incubated DNA hybridization surface to produce a rinsed incubated DNA hybridization surface; (c) contacting the rinsed incubated DNA hybridization surface with a liquid crystal; and (d) determining whether the liquid crystal is uniformly anchored on the rinsed incubated DNA hybridization surface.

### CROSS-REFERENCES TO RELATED APPLICATIONS

This application is a divisional of U.S. Ser. No. 10/262,562, filed Oct. 1, 2002, which claims priority to U.S. Provisional Application No. 60/327,138, filed on Oct. 4, 2001, the entire disclosure of which is incorporated herein by reference for all purposes.

### GOVERNMENT RIGHTS

This invention was made with United States government support awarded by the following agency: NAVY N00014-99-0250. The United States has certain rights in this invention.

### FIELD OF THE INVENTION

The invention relates generally to methods and devices for detecting DNA hybridization. More particularly, the invention relates to methods and devices for detecting DNA hybridization using liquid crystals and nucleic acid sequences bound to a surface.

### BACKGROUND OF THE INVENTION

Methods for detecting the presence of biological substances and chemical compounds in samples has been an area of continuous development in the field of analytical chemistry and biochemistry. Various methods have been developed that allow for the detection of various target species in samples taken from sources such as the environment or a living organism. Detection of a target species is often necessary in clinical situations before a prescribed method of treatment may be undertaken and an illness diagnosed. DNA is just one example of a target species of interest, and the ability to detect a complementary strand of DNA or a fragment of DNA is of particular importance. The ability to confirm the presence of a complementary strand of DNA or a fragment of DNA has application in a wide variety of areas including criminology, forensics, tissue typing, and genomics.

Several types of assay currently exist for detecting the presence of target species in samples. One conventional type of assay is the radioimmunoassay (RIA). RIA is a highly sensitive technique that can detect very low concentrations of antigen or antibody in a sample. RIA involves the competitive binding of radiolabeled antigen and unlabeled antigen to a high-affinity antibody. By measuring the amount of labeled antigen free in solutions, it is possible to determine the concentration of unlabeled antigen. Kuby, J., Immunology, W.H. Freeman and Company, New York, N.Y. (1991), pp. 147-150.

Another type of assay which has become increasingly popular for detecting the presence of pathogenic organisms is the enzyme-linked immunosorbent assay or ELISA. This type of assay allows pathogenic organisms to be detected using biological species capable of recognizing epitopes associated with proteins, viruses and bacteria. Generally, in an ELISA assay, an enzyme conjugated to an antibody will react with a colorless substrate to generate a colored reaction product if a target species is present in the sample. Kuby, J., Immunology, W.H. Freeman and Company, New York, N.Y. (1991), pp. 147-150. Physically adsorbed bovine serum albumin has been used in various such assays as a blocking layer because it has been found to prevent the non-specific adsorption of biological species that might interfere with or result in erroneous assay results.

Although ELISA and other immunosorbent assays are simple and widely used methods, they have several disadvantages. Tizard, I. R. Veterinary Immunology: An Introduction, W.B. Saunders Company, Philadelphia, Pa. (1996); Harlow, Ed.; Lane, D. Antibodies: A Laboratory Manual, Cold Spring Harbor Laboratory, Cold Springs Harbor, N.Y. (1988); Van Oss, C. J.; van Regenmortel, M. H. V. Immunochemistry, Dekker, New York, N.Y. (1994). Labeled antibodies can be expensive, especially for assays requiring radioactive labels. Additionally, radioactive labels require special handling as radioactive materials are also hazardous. The labeling of a compound, which is the main drawback of these methods, may alter the binding affinity of antibody to analyte.

Qualitative diagnostic assays based on aggregation of protein coated beads have been used for detecting proteins and viruses. Tizard, I. R. Veterinary Immunology: An Introduction, W.B. Saunders Company, Philadelphia, Pa. (1996): Cocchi, J. M.; Trabaud, M. A.; Grange, J.; Serres, P. F.; Desgranges, C. J. Immunological Meth., 160, (1993), pp. 1; Starkey, C. A.; Yen-Lieberman, B.; Proffitt, M. R. J. Clin. Microbiol., 28, (1990), pp. 819; Van Oss, C. J.; van Regenmortel, M. H. V. Immunochemistry, Dekker, New York, N.Y. (1994). For direct detection of antibodies, antigen is non-specifically adsorbed to the surface of latex beads which are several microns in diameter. The protein-coated beads possess a slight charge which prevents aggregation. Introduction of an antibody specific to the adsorbed protein can link the beads, leading to agglutination.

To overcome the need for labeled proteins, principles based on direct detection of the binding of proteins and ligands have been investigated. Schmitt, F.-J.; Haussling, L.; Ringsdorf, H.; Knoll, W. Thin Solid Films, 210/211, (1992), pp. 815; Hauslling, L.; Ringsdorf, H. Langmuir, 7, (1991), pp. 1837. Surface plasmon reflectometry (SPR) is one such method. SPR is sensitive to changes in the index of refraction of a fluid near a thin metal surface that has been excited by evanescent electromagnetic waves. Typical angular resolution using this method is 0.005° allowing detection of sub-angstrom changes in adsorbed film thickness with SPR. A thermally stable environment is required due to the dependence of the resonance angle on the index of refraction of the fluid.

The use of ion-channel switches for detecting biospecific interactions has also been reported. Cornell, B. A.; Braach-Maksvytis, V. L. B.; King, L. G.; Osman, P. D. J.; Raguse, B.; Wieczorek, L.; Pace, R. J. Nature, 387, (1997), pp. 580. In a device using ion channel switches, a tethered lipid membrane incorporating mobile ion channels is separated from a gold electrode surface by an ion reservoir. The gold surface serves as an anchor for the membrane and acts as an electrode. Within the membrane are upper and lower ion channels. In order to become conductive, the outer and inner ion channels must align and form a dimer. Membrane spanning lipids, which help stabilize the lipid membrane, are attached at one end to the electrode surface and are terminated with ligands that extend away from the membrane. The ion channels of the outer layer possess ligands. This method requires sensitive devices for detecting the change in conductance.

A method based on a porous silicon support that permits optical detection of the binding of specific proteins to ligands has been reported. Lin, V.; Motesharei, K.; Dancil, K. S.; Sailor, M. J.; Ghadiri, M. R. Science, 278, (1997), pp. 840; Dancil, K. S.; Greiner, D. P.; Sailor M. J. J. Am. Chem. Soc., 121, (1999), pp. 7925. The porous areas are typically 1 to 5 μm deep and a few square micrometers to millimeters in area. Typical binding times are on the order of 30 minutes followed by rinsing of the surface. Initial work in this area incorrectly reported the detection of extremely low concentrations of analyte. Binding of streptavidin to biotinylated surfaces was initially found to reduce the index of refraction of the porous support, however this was later correctly attributed to surface oxidation. In addition, a change in the effective optical thickness of the film was reportedly observed upon introduction of streptavidin, however, differentiation between specific interactions and non-specific adsorption could not be made. This method does not require labeled molecules, however, the porous silicon surface is susceptible to oxidation and non-specific adsorption.

The use of polymerized multilayer assemblies for the detection of receptor-ligand interactions has also been reported. Charych, D. H.; Nagy, J. O.; Spevak, W.; Bednarski, M. D. Science, 261, (1993), pp. 585; Pan, J. J.; Charych, D. Langmuir, 13, (1997), pp. 1365. Polydiacetylene multilayer films deposited by Langmuir-Blodgett technique change color from blue to red due to a conformational change in the polymer backbone. The response can be controlled and used for protein detection by attaching ligands to the multilayer. Upon binding of a multivalent macromolecule to ligands, stress is introduced into the multilayer assembly. A change in color is seen in the system if sufficient protein is bound, with binding times typically on the order of 30 minutes. This system permits direct detection of receptor-ligand interactions and transduces the events into an optical signal that can be easily measured and quantified. The optical output can be interpreted by eye or analyzed with a spectrophotometer for quantitative conclusions. The use of polymerized multilayer assemblies for the detection of influenza virus has been demonstrated.

Although many of the conventional assay methods described above work well in detecting the presence of target species, many conventional assay methods are expensive and often require instrumentation and highly trained individuals, which makes them difficult to use routinely in the field. Thus, a need exists for assay devices and systems which are easier to use and which allow for evaluation of samples in remote locations.

Recently, assay devices that employ liquid crystals have been disclosed. For example, a liquid crystal assay device using mixed self-assembled monolayers (SAMs) containing octanethiol and biotin supported on an anisotropic gold film obliquely deposited on glass has recently been reported. Gupta, V. K.; Skaife, J. J.; Dubrovsky, T. B., Abbott N. L. Science, 279, (1998), pp. 2077-2079. In addition, PCT publication WO 99/63329 published on Dec. 9, 1999, discloses assay devices using SAMs attached to a substrate and a liquid crystal layer that is anchored by the SAM. U.S. Pat. No. 6,288,392 issued to Abbott et al. discloses the quantitative characterization of obliquely-deposited substrates of gold using atomic force microscopy and describes the influence of substrate topography on the anchoring of liquid crystals. U.S. Pat. No. 6,284,197 issued to Abbott et al. discloses the optical amplification of molecular interactions using liquid crystals.

Lyotropic water-based liquid crystals have been reported as a useful amplification system in the detection of certain biological materials, but not DNA, in PCT publication WO 99/64862 published on Dec. 16, 1999. A diluting solvent, water, is used in conjunction with a surfactant, cetylpyridinium chloride, to change the concentration of a solid crystal and create the lyotropic liquid crystal. Ligand-specific receptors are incorporated in the lyotropic liquid crystal. Binding of a ligand such as a microbe to a ligand-specific receptor such as an antibody in the lyotropic liquid crystal purportedly distorts the lyotropic liquid crystal inducing birefringence with concomitant generation of detectable light. In the PCT publication, lyotropic liquid crystals are reported as superior to other types of liquid crystals for detection of biological molecules because the lyotropic liquid crystals readily incorporate the ligand-specific receptors.

Although various methods have been used to detect DNA hybridization, a need exits for a simple device and method that may be used to rapidly detect the presence of complementary strands of DNA and DNA fragments or nucleic acid sequences in a sample without the need for labeling and without the need for complex instrumentation such as surface plasmon reflectometry. A need also remains for a method of manufacturing a device for use in detecting the presence of complementary strands of DNA and DNA fragments or nucleic acid sequences in a sample.

### SUMMARY OF THE INVENTION

The present invention provides devices and methods for detecting the presence of DNA or a strand of nucleic acids in a sample. The invention also provides a method for preparing a device for detecting DNA hybridization on a surface.

A method for preparing a surface for use in detecting DNA hybridization in a sample is provided that includes: rinsing a DNA hybridization surface with at least one rinsing solution to produce a rinsed DNA hybridization surface. The DNA hybridization surface includes a support with a self-assembled monolayer adsorbed on a metallized surface. The self assembled monolayer includes an alkanethiol and includes a strand of nucleic acids having a functional group that binds to the metallized surface of the support.

In some methods for preparing a surface for use in detecting DNA hybridization in a sample, the method includes contacting the metallized surface of the support with the alkanethiol and the strand of nucleic acids that includes the functional group that binds to the metallized surface to provide the DNA hybridization surface. In some such methods, the alkanethiol and the strand of nucleic acids having the functional group that binds to the metallized surface of the support are in one solution and are contacted with the metallized surface of the support at the same time. In other such methods of preparing a surface for use in detecting DNA hybridization in a sample, the alkanethiol is in a first solution and the strand of nucleic acids comprising the functional group that binds to the metallized surface is in a second solution. In some such methods, the first solution is contacted with the metallized surface of the support and then the second solution is contacted with the metallized surface of the support. In other such methods, the second solution is contacted with the metallized surface of the support and then the first solution is contacted with the metallized surface of the support. In some methods in which the alkanethiol is in a first solution and the strand of nucleic acids having the functional group that binds to the metallized surface is in a second solution, the second solution comprising the strand of nucleic acids having the functional group that binds to the metallized surface is a phosphate buffered aqueous solution comprising the strand of nucleic acids having the functional group that binds to the metallized surface at a concentration ranging from about 0.01 μM to about 10 mM.

In some methods of preparing a surface for use in detecting DNA hybridization in a sample, the support comprises a top layer of a metal such as gold providing the metallized surface. In some embodiments, the metal such as gold is obliquely deposited at an angle ranging from 30° to about 60° to a planar surface of the support. In other embodiments, the top layer of the metal such as gold has a thickness ranging from 50 Å to 300 Å (from 5 nm to 30 nm).

In some methods of preparing a surface for use in detecting DNA hybridization in a sample, the top layer of metal providing the metallized surface is deposited on the support over a layer of a material that promotes the adhesion of the metal such as gold. In some such methods, the material that promotes adhesion is titanium, and in some such methods, the titanium is present on the support in a layer with a thickness ranging from 5 Å to 100 Å (from 0.5 nm to 10 nm). In other such methods, the titanium is present on the support in a layer with a thickness ranging from 5 Å to 20 Å (from 0.5 nm to 2 nm).

Other methods are provided in which at least two rinsing solutions are used to form the rinsed DNA hybridization surface. In some such methods, at least one of the two rinsing solutions is a phosphate buffered aqueous solution, a Tris buffered aqueous solution, or a sodium chloride solution that includes phosphate or Tris, and at least one of the two rinsing solutions is water, an alcohol, or a combination of water and an alcohol. In some such methods, the DNA hybridization surface is first rinsed with the phosphate buffered aqueous solution and is then rinsed with the water, the alcohol, or the combination of the water and the alcohol. In some such methods, the DNA hybridization surface is first rinsed with the phosphate buffered aqueous solution and is then rinsed with distilled or deionized water. In other such methods, the DNA hybridization surface is first rinsed with the phosphate buffered aqueous solution and is then rinsed with an alcohol such as ethanol or methanol.

In some methods of preparing a surface for use in detecting DNA hybridization in a sample, the at least one rinsing solution is selected from water, an alcohol, or mixtures thereof. In other methods, the at least one rinsing solution is deionized or distilled water. In still other methods, the at least one rinsing solution is ethanol or methanol.

In some methods of preparing a surface for use in detecting DNA hybridization in a sample, the self assembled monolayer of the DNA hybridization surface has a thickness ranging from 5 Å to 300 Å (from 0.5 nm to 30 nm) as determined by ellipsometry.

In other methods of preparing a surface for use in detecting DNA hybridization in a sample, the functional group that binds to the metallized surface of the strand of nucleic acids is a thiol group.

In still other methods of preparing a surface for use in detecting DNA hybridization in a sample, the strand of nucleic acids having the functional group includes 5 to 200 nucleic acids and in other methods includes 10 to 40 nucleic acids.

In still other methods of preparing a surface for use in detecting DNA hybridization in a sample, the alkanethiol has 4 to 20 carbon atoms. In some such methods, the alkanethiol is hexanethiol.

A device for detecting DNA hybridization in a sample is also provided. The device includes a support having a metallized surface that has a top surface with an alkanethiol and a strand of nucleic acids with a functional group such as a thiol group that binds to the metallized surface adsorbed on it. The alkanethiol and the strand of nucleic acids form a self assembled monolayer. The top surface of the device is preferably a rinsed surface such that the surface is substantially free of excess sodium salts, potassium salts, and Tris salts.

Other devices for detecting DNA hybridization in a sample are provided which have any of the additional features described in the preceding paragraphs such as with respect to the metallized surface, the adhesion promoting material, the strand of nucleic acids with the functional group that binds to the metallized surface, the alkanethiol, or any combination of these.

A method for detecting DNA hybridization is also provided. The method includes: (a) incubating a DNA hybridization surface with an aqueous sample that includes a fragment of DNA to produce an incubated DNA hybridization surface; (b) rinsing the incubated DNA hybridization surface to produce a rinsed incubated DNA hybridization surface that is, in some embodiments, substantially free of excess sodium salts, potassium salts, and Tris salts; (c) contacting the rinsed incubated DNA hybridization surface with a liquid crystal; and (d) determining whether a uniform anchoring of liquid crystal has been disrupted on the rinsed incubated DNA hybridization surface. In such methods, the DNA hybridization surface includes a support that includes a self assembled monolayer on a metallized surface of the support. The self-assembled monolayer includes an alkanethiol and includes a strand of nucleic acids having a functional group that binds to the metallized surface of the support. A change in the anchoring of the liquid crystal on the rinsed incubated DNA hybridization surface compared to the anchoring of the liquid crystal on the DNA hybridization surface prior to incubation indicates that the strand of DNA in the aqueous sample is complementary to the strand of nucleic acids of the self assembled monolayer. In some methods, a disruption in the uniform anchoring of the liquid crystal on the rinsed incubated DNA hybridization surface indicates that the strand of DNA in the aqueous sample is complementary to the strand of nucleic acids of the self assembled monolayer.

Other methods for detecting DNA hybridization are provided which have any of the additional features with respect to the method for preparing a surface for use in detecting DNA hybridization in a sample.

Other methods for detecting DNA hybridization are provided in which the DNA hybridization surface is rinsed with deionized water, distilled water, an alcohol, or any combination of these after it has been incubated with the aqueous solution sample.

Still other methods for detecting DNA hybridization are provided in which the aqueous sample that includes the fragment of DNA also includes tris(hydroxymethyl)amine, ethylenediaminetetraacetic acid, sodium chloride, sodium or potassium phosphate, or combinations of these.

Yet other methods for detecting DNA hybridization are provided in which the DNA hybridization surface is incubated with the aqueous solution sample at a temperature ranging from 20° C. or about 20° C. to 60° C. or about 60° C., from 20° C. or about 20° C. to 40° C. or about 40° C., from 22° C. or about 22° C. to 28° C. or about 28° C., or 25° C. or about 25° C.

Still further methods for detecting DNA hybridization are provided in which the DNA hybridization surface is incubated with the aqueous solution sample for a period of time ranging from 1 hour to 24 hours.

Yet other methods for detecting DNA hybridization are provided in which the liquid crystal is a nematic liquid crystal. In still other methods, the liquid crystal is 4-cyano-4′-pentylbiphenyl.

Another method for detecting DNA hybridization is provided. The method includes: (a) depositing titanium on a top surface of a glass support to provide a layer of titanium with a thickness ranging from 5 Å or about 5 Å to 20 Å or about 20 Å (ranging from 0.5 nm or about 0.5 nm to 2 nm or about 2 nm); (b) obliquely depositing a metal such as gold on top of the layer of titanium to provide a support with a metallized surface that includes a top layer of gold with a thickness ranging from 50 Å or about 50 Å to 300 Å or about 300 Å (ranging from 5 nm or about 5 nm to 30 nm or about 30 nm); (c) contacting a top surface of the metallized surface of the support with a solution that includes an alcohol such as ethanol or methanol and hexanethiol at a concentration of from about 0.5 mM to about 1.0 mM for at least one hour and contacting an aqueous KH₂PO₄ buffered solution that includes a strand of nucleic acids having a thiol group at a concentration of from about 0.01 μM to about 10 mM for a first period of time of from 30 minutes or about 30 minutes to 120 minutes or about 120 minutes to provide a DNA hybridization surface; (d) incubating the DNA hybridization surface for a second period of time ranging from 1 hour or about 1 hour to 24 hours or about 24 hours at a temperature ranging from 20° C. or about 20° C. to 40° C. or about 40° C. with an aqueous solution sample that includes a fragment of DNA, and additionally includes tris(hydroxymethyl)amine, ethylenediaminetetraacetic acid, sodium chloride, sodium or potassium phosphate, or combinations of these to provide an incubated DNA hybridization surface; (e) rinsing the incubated DNA hybridization surface with deionized water, distilled water, ethanol, or combinations of these to provide a rinsed incubated DNA hybridization surface; (f) contacting the top surface of the rinsed incubated DNA hybridization surface produced in (e) with a liquid crystal such as a nematic liquid crystal such as 4-cyano-4′-pentylbiphenyl; and (g) determining whether the anchoring of the liquid crystal on the rinsed incubated DNA hybridization surface has changed compared to the anchoring of the liquid crystal on the DNA hybridization surface prior to incubation. A change in the anchoring of the liquid crystal indicates DNA hybridization has occurred. In some such methods, a disruption in the uniform anchoring of the liquid crystal on the rinsed incubated DNA hybridization surface indicates that DNA hybridization has occurred.

Kits and optical cells for detecting DNA hybridization in a sample are also provided. Such kits and optical cells may have any of the features described herein.

Further objects, features and advantages of the invention will be apparent from the following detailed description when taken in conjunction with the accompanying drawings.

### BRIEF DESCRIPTION OF THE DRAWINGS

FIG. 1 is a cross sectional schematic diagram of a DNA hybridization surface with an optional adhesion promoting layer.

FIGS. 2a-2d are scanned images showing the optical textures of optical cells prepared from glass slides with obliquely deposited gold after immersion in an ethanolic hexanethiol solution for 60 minutes at 37° C., but with increasing immersion times in aqueous solutions containing 5′-HS—(CH₂)₆-TGC-AGT-TCC-GGT-GGC-TGA-TC-3′ at a concentration of 0.5 μM and at 37° C. (FIG. 2a, 0.5 hours; FIG. 2b, 1.5 hours; FIG. 2c, 2.5 hours; and FIG. 2d, 24 hours).

FIG. 3 is a graph showing the ellipsometric thicknesses (Å) of the DNA and alkanethiol on the glass slides used to prepare the optical cells of FIGS. 2a-2d as a function of immersion time in the DNA fragment adsorption solution.

FIGS. 4a-4d are scanned images showing the optical textures of optical cells prepared from glass slides with obliquely deposited gold after immersion in an ethanolic hexanethiol solution for 60 minutes at 37° C., but with increasing immersion times in aqueous solutions containing 5′-HS—(CH₂)₆-TGC-AGT-TCC-GGT-GGC-TGA-TC-3′ at a concentration of 0.5 μM and at 25° C. (FIG. 4a, 0.5 hours; FIG. 4b, 1.5 hours; FIG. 4c, 2.5 hours; and FIG. 4d, 24 hours).

FIG. 5 is a graph showing the ellipsometric thicknesses (Å) of alkanethiol and DNA on the glass slides used to prepare the optical cells of FIGS. 4a-4d as a function of immersion time in the DNA fragment adsorption solution.

FIGS. 6a-6e are scanned images showing the optical textures of optical cells prepared from glass slides with obliquely deposited gold after initial immersion for 0.0 minutes (FIG. 6a), 0.5 minutes (FIG. 6b), 3 minutes (FIG. 6c), 5 minutes (FIG. 6d), and 48 hours (FIG. 6e) in aqueous solutions containing 5′-HS—(CH₂)₆-TGC-AGT-TCC-GGT-GGC-TGA-TC-3′ at a concentration of 1.0 μM at 25° C. and then immersion in an ethanolic hexanethiol solution for 60 minutes at 25° C. The glass slide used to prepare scanned image FIG. 6e was not immersed in the alkanethiol solution.

FIG. 7 is a graph showing the thicknesses (Å) measured using ellipsometry (first bar) and x-ray photoelectron spectroscopy (Au 4f_{7/2}) (second bar) of the alkanethiol and DNA on the glass slides used to prepare the optical cells of FIGS. 6a-6e as a function of immersion time in the DNA fragment adsorption solution.

FIG. 8 is a graph showing the nitrogen peak height measured using x-ray photoelectron spectroscopy (N) of the glass slides used to prepare the optical cells of FIGS. 6a-6e as a function of immersion time in the DNA fragment adsorption solution.

FIGS. 9a-9d are scanned images of the optical textures of optical cells made from DNA hybridization surfaces prepared under identical DNA fragment adsorption and alkanethiol adsorption conditions. The DNA hybridization surfaces were rinsed with an aqueous solution of TE (FIG. 9a), deionized water (FIG. 9b), with an aqueous solution of TE and then with deionized water (FIG. 9C), or with an aqueous solution of TE and then with ethanol (FIG. 9d).

FIGS. 10a and 10b are scanned images showing the optical textures of optical cells prepared from DNA hybridization surfaces after incubation in aqueous TE solutions without (FIG. 10a) and with (FIG. 10b) a complementary target DNA fragment.

FIGS. 11a and 11b are scanned images showing the optical textures of optical cells prepared from DNA hybridization surfaces different than those in FIGS. 10a and 10b after incubation in aqueous TE solutions without (FIG. 11a) and with (FIG. 11b) a complementary target DNA fragment.

FIGS. 12a and 12b are scanned images showing the optical textures of optical cells prepared from DNA hybridization surfaces different from those in FIGS. 10a, 10b, 11a, and 11b after incubation in aqueous TE solutions without (FIG. 12a) and with (FIG. 12b) a complementary target DNA fragment.

FIGS. 13a and 13b are scanned images of the optical textures of optical cells prepared from slides without any bound DNA fragment showing that target DNA does not adsorb on the surface in the absence of the bound complementary DNA fragment. FIG. 13a is a scanned image of the optical texture of an optical cell prepared from a slide immersed in an aqueous solution of TE, and FIG. 13b is a scanned image of the optical texture of an optical cell prepared from a slide immersed in an aqueous solution of TE containing a fragment of DNA that did not contain a functional group that binds to the metallized surface.

FIGS. 14a and 14b are scanned images of the optical textures of optical cells prepared from glass slides with obliquely deposited gold on them which were immersed in ethanolic solutions of hexanethiol for 60 minutes at 37° C. and then for 30 minutes at 25° C. in an aqueous solutions containing 5′-HS—(CH₁₂)₆-TGC-AGT-TCC-GGT-GGC-TGA-TC-3′ at a concentration of 0.5 μM. FIG. 14a is a scanned image obtained from the slide prepared as described above, and FIG. 14b is a scanned image obtained from a slide prepared as above after incubation for 3 hours in an aqueous TE buffered solution containing 5′-GAT-CAG-CCA-CCG-GAA-CTG-CA-3′ at a concentration of 1 mM and a temperature of 25° C.

FIGS. 15a and 15b are scanned images of the optical textures of optical cells prepared from glass slides with obliquely deposited gold on them which were immersed in ethanolic solutions of hexanethiol for 60 minutes at 37° C. and then for 30 minutes at 25° C. in an aqueous solutions containing 5′-HS—(CH₂)₆-TGC-AGT-TCC-GGT-GGC-TGA-TC-3′ at a concentration of 0.5 μM. FIG. 15a is a scanned image obtained from the slide prepared as described above, and FIG. 15b is a scanned image of the slide prepared as above after incubation for 24 hours in an aqueous TE buffered solution containing 5′-GAT-CAG-CCA-CCG-GAA-CTG-CA-3′ at a concentration of 1 μM and a temperature of 25° C.

### DETAILED DESCRIPTION OF THE INVENTION

Generally, the invention provides devices and methods for detecting DNA in a sample. The invention also generally provides methods for preparing devices for detecting DNA hybridization on a surface.

The symbols “A”, “T”, “C”, “G”, and “U” as used herein respectively refer to the nucleotide bases adenine, thymine, cytosine, guanine, and uracil.

The phrase “DNA recognition fragment” refers to a strand of DNA or a fragment of a strand of DNA such as a strand of nucleic acids that is bound to a metallized surface of a support in a DNA hybridization surface. The “DNA recognition fragment” bound to the metallized surface of the support is capable of recognizing and binding a strand or a fragment of a strand of complementary target DNA or nucleic acids resulting in hybridization. The hybridization gives rise to a visual appearance of the LC that is distinct from that observed in the absence of hybridization. The change in the appearance of the liquid crystal could be between two disordered states or two ordered states. In some embodiments, the change in the appearance of the liquid crystal due to hybridization could result from disruption in the ability of the DNA hybridization surface to uniformly anchor the liquid crystal such that the change observed is from ordered anchoring of liquid crystals to disordered anchoring of liquid crystals.

A DNA hybridization surface is “substantially free” of excess sodium salts, potassium salts, and Tris salts if it has been rinsed with water, an alcohol, or a combination of these after the metallized surface used to prepare the DNA hybridization surface has been contacted with a solution containing an alkanethiol and a solution containing a DNA recognition fragment or a solution containing both an alkanethiol and a DNA recognition fragment.

An incubated DNA hybridization surface and a rinsed incubated DNA hybridization surface are “substantially free” of excess sodium salts, potassium salts, and Tris salts if they have been rinsed with water, an alcohol, or a combination of these after a DNA hybridization surface has been contacted with an aqueous solution sample comprising a fragment of DNA.

The term “Tris” refers to tris(hydroxymethyl)aminomethane.

The acronym “EDTA” refers to ethylenediaminetetraacetic acid.

The acronym “TE” refers to an aqueous solution containing 10 mM Tris, 1 mM EDTA, and 1 M sodium chloride at a pH of 7.

The term “about” as used herein in conjunction with a number refers to a range of from 95% to 105% of that number. For example a temperature of about 60° C. refers to a temperature ranging from 57° C. to 63° C.

All ranges recited herein include all combinations and subcombinations included within that range's limits. For example, a temperature range of from about 20° C. to about 65° C. includes ranges of from 20° C. to 60° C., of from 25° C. to 30° C., of from 25° C. to 28° C., and of from 20° C. to 30° C., etc. Similarly an ellipsometric thickness range of from about 10 Å (1 nm) to about 25 Å (2.5 nm) includes ranges of from 10 Å (1 nm) to 20 Å (2 nm), of from 12 Å (1.2 nm) to 16 Å (1.6 nm), of from 15 Å (1.5 nm) to 20 Å (2.0 nm), and of from 13 Å (1.3 nm) to 18 Å (1.8 nm), etc. Furthermore, one skilled in the art will recognize that any listed range can be easily recognized as sufficiently describing and enabling the same range being broken down into at least equal halves, thirds, quarters, fifths, tenths, etc. As a non-limiting example, each range discussed herein can be readily broken down into a lower third, middle third, and upper third.

Some of the characteristics which a suitable DNA hybridization surface should possess include: the ability to resist non-specific adsorption; the ability to orient liquid crystals in a reproducible manner; and the possession of anisotropic structure that the specific binding of a complementary strand of DNA or a fragment of a strand of DNA can alter or partially or completely erase. The latter characteristic drives a change in the anchoring of liquid crystals which indicates that the target species is present in the sample.

A wide variety of materials may be used as supports to prepare the DNA hybridization surface in the devices and methods of the present invention as will be apparent to those skilled in the art. Preferred supports include polymers and silica-containing materials. Examples of polymeric supports include, but are not limited to, polystyrene, polycarbonates, and polymethyl methacrylate. Other materials suitable for use as supports include metal oxides such as, but not limited to, indium oxide, tin oxide, and magnesium oxide and metals such as, but not limited to, gold, silver, and platinum. Still other materials that may be used as supports include cellulosic materials such as nitrocellulose, wood, paper, and cardboard, and sol-gel materials. In some embodiments, supports include glass, quartz, and silica, or more preferably, glass slides, glass plates, and silica wafers. Preferably, such supports are cleaned prior to use. For example, glass slides and plates are preferably cleaned by treatment in “piranha solution” (70% H₂SO₄/30% H₂O₂) for 1 hour and then rinsed with deionized water before drying under a stream of nitrogen. “Piranha solution” requires care in handling as it reacts violently with organic compounds and should not be stored in closed containers.

A preferred support in accordance with the present invention contains a top surface with a layer of obliquely deposited metal on it. Metals that may be used include, but are not limited to, gold, silver, copper, platinum, and palladium. Optionally, an obliquely deposited metal surface such as a gold or silver surface will overlay a surface of titanium or other material that promotes adhesion which has already been deposited on a top surface of the support. The use of the titanium provides better adhesion of the obliquely deposited metal such as silver, or more preferably gold in preparing the metallized surface. Chromium and organic adhesion promoters, such as, but not limited to, aminopropyltrialkoxysilanes may also be utilized in accordance with the present invention. The use of titanium or another adhesion-promoting material is not required as suitable DNA hybridization surfaces may be prepared without the use of such materials. If an adhesion promoting material is used, a layer of varying thickness may be applied to the underlying support. In some embodiments, approximately 10 Å of Ti is deposited on a support such as a glass slide or plate. In other embodiments, the amount of adhesion-promoting material ranges from 5 Å (0.5 nm) or about 5 Å (0.5 μm) to 20 Å (2.0 nm) or about 20 Å (2.0 nm) while in other embodiments the thickness ranges from 8 Å (0.8 nm) or about 8 Å (0.8 nm) to 15 Å (1.5 nm) or about 15 Å (1.5 nm). In some embodiments, approximately 10 Å (1.0 nm) of aminopropyltrimethoxy-silane is deposited as an adhesion-promoting material. In other embodiments, the thickness of the layer of adhesion promoting material ranges from 5 Å (0.5 nm) or about 5 Å (0.5 nm) to 50 Å (5 nm) or about 50 Å (5 mm). The amount of adhesion-promoting material may be thicker such that in some embodiments, the thickness of the layer of an adhesion-promoting material such as titanium ranges from 5 Å (0.5 nm) or about 5 Å (0.5 nm) to 100 Å (10 nm) or about 100 Å (10 nm).

In some embodiments, a layer of an obliquely deposited metal, preferably gold, is deposited on a cleaned surface of the support by evaporating it at a rate of about 0.2 Å/s (0.02 nm/s) at a pressure of less than or about 5×10⁻⁶ torr without rotation of the sample relative to the incident flux of gold. See Gupta, V. K. et al. Chemistry of Materials, 8, (1996), p. 1366. In other embodiments, a metal such as gold is deposited as described above on a top surface of a support that contains an adhesion-promoting material such as titanium. The layer of a metal such as gold on the metallized surface of the support typically ranges from 50 Å (5 nm) or about 50 Å (5 nm) to 300 Å (30 nm) or about 300 Å (30 nm) in thickness. In other embodiments, the layer of a metal such as gold deposited on the surface of the support ranges from 80 Å (8 nm) or about 80 Å (8 nm) to 250 Å (25 nm) or about 250 Å (25 nm) in thickness or from 90 Å (9 nm) or about 90 Å (9 nm) to 200 Å (20 nm) or about 200 Å (20 nm) in thickness. In still other embodiments, the layer of the metal such as gold deposited on the support is from 100 Å (10 nm) or about 100 Å (10 nm) to 200 Å (20 nm) or about 200 Å (20 nm). In some embodiments, a metal such as gold is deposited at an angle of from 30° or about 30° to 60° or about 60°. In other preferred embodiments, a metal such as gold is deposited at an angle of 50° or about 50°. The angle at which the gold is deposited on an underlying support has been found to impact the sensitivity of the DNA hybridization surface. Therefore, different angles of metal deposition may be preferred depending on the particular application as will be apparent to those skilled in the art. The metallized surface obtained after deposition of the metal is generally an anisotropically rough and semi-transparent surface.

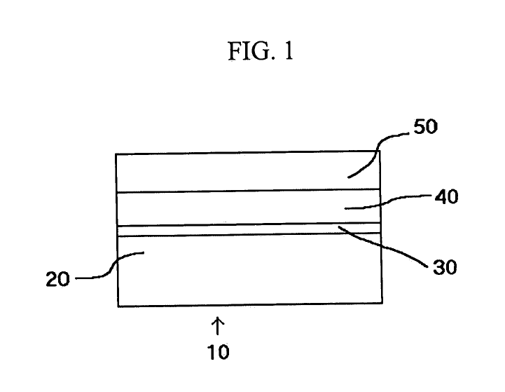

FIG. 1 is a cross-sectional schematic representation of a DNA hybridization surface 10 with an optional layer of adhesion promoting material 30 deposited over support 20. As shown in FIG. 1, a metal layer 40 is deposited over the layer of adhesion promoting material 30. Self-assembled monolayer 50 includes an alkanethiol and includes a strand of nucleic acids with a functional group that binds to the metallized surface on the top of metal layer 40.

The DNA hybridization surface includes an alkanethiol and a DNA recognition fragment such as a strand of nucleic acids that are adsorbed on the metallized surface of the support. The alkanethiol may be adsorbed on the metallized surface from a solution that includes both the DNA recognition fragment and the alkanethiol. In this manner, both the DNA recognition fragment and the alkanethiol will be adsorbed on the metallized surface at the same time using the same solution. In another method, the alkanethiol is first adsorbed on the metallized surface from one solution, and then the DNA recognition fragment is adsorbed on the metallized surface from another solution containing the DNA recognition fragment. In yet another method, the DNA recognition fragment is first adsorbed on the metallized surface of the support and then the alkanethiol is adsorbed on the metallized surface. Each of the above methods has been found useful in preparing suitable DNA hybridization surfaces for detecting DNA hybridization.

As noted above, in some embodiments the DNA hybridization surfaces are prepared by adsorbing an alkanethiol on a surface of a support that contains the obliquely deposited gold or silver (the metallized surface). This is typically accomplished by immersing the support with the obliquely deposited gold, silver, or other metal in a solution containing the alkanethiol. Alternatively, a solution may be dropped or poured onto the surface or otherwise contacted with the surface of the support containing the metal. The thiol (—SH) group of the alkanethiol binds to the metal on the support immobilizing the alkanethiol on the surface. As noted above, the alkanethiol is adsorbed onto the surface of the support from a solution containing the alkanethiol. In some embodiments, the alkanethiol is present in an alcohol such as ethanol or methanol although other liquids may also be employed in accordance with the invention.

Various alkanethiols may be used to prepare DNA hybridization surfaces. Suitable alkanethiols include, but are not limited to, C₄ to C₂₀ alkanethiols such as butanethiol, pentanethiol, hexanethiol, heptanethiol, octanethiol, nonanethiol, decanethiol, undecanethiol, dodecanethiol, tridecanethiol, tetradecanethiol, pentadecanethiol, hexadecanethiol, heptadecanethiol, octadecanethiol, nonadecanethiol, and eicosanethiol. In various embodiments, the alkanethiols include C₅ to C₁₂ alkanethiols, C₅ to C₁₀ alkanethiols, C₅ to C₈ alkanethiols, or hexanethiol. Those skilled in the art will recognize that dialkyl disulfides, R—S—S—R, may also be used to prepare DNA hybridization surfaces. Omega-functionalized alkanethiols may also be used and are encompassed in the group of compounds referred to as “alkanethiols”. For example, mercaptohexanol may be used in place of or with hexanethiol to prepare self assembled monolayers in one embodiment of the invention. Examples, include omega groups of hydroxyl, nitrile, carboxylic acid, ethylene oxide, diethylene oxide, triethylene oxide, tetraethylene oxide, pentaethylene oxide, or polyethylene oxide. In one embodiment, the omega group is the hydroxyl group with alkanethiol chain lengths ranging from C₄ to C₂₀, and in some embodiments C₆.

The concentration of the alkanethiol in the solution used for alkanethiol adsorption generally ranges from about 1 micromolar to 10 millimolar. When using 1 micromolar solutions, preferred immersion times range from 10 seconds to 24 hours. Particularly preferred immersion times range from 1 minute to 6 hours. Other preferred immersion times range from 30 minutes to 2 hours. Typically, DNA hybridization surfaces were prepared by contacting metallized surfaces of a support with an ethanolic solution of an alkanethiol such as hexanethiol at a concentration of 1 mM for a period of at least about 1 hour. Longer or shorter contact times may be used as long as a densely packed monolayer is obtained as will be apparent to those of skill in die art. Generally, the lower the concentration of the alkanethiol in the alkanethiol solution, the longer the metallized surface will be contacted with the alkanethiol solution. Conversely, the higher the concentration of the alkanethiol in the alkanethiol solution, the shorter the metallized surface will be contacted with the alkanethiol.

The alkanethiols are typically adsorbed onto the metallized surface of the support in solutions at temperatures ranging from about 15° C. to about 60° C., from about 20° C. to about 40° C., from about 22° C. to about 40° C., or from about 25° C. to about 37° C. In some embodiments, the temperature range is from about 22° C. to about 28° C., and in other embodiments the temperature is about 25° C. A steady temperature is not necessary, and the temperature may be increased or decreased during the alkanethiol adsorption. Generally, the temperature of the alkanethiol solution is not critical to the preparation of the DNA hybridization surface. If the DNA recognition fragment has previously been adsorbed onto the metallized surface of the support, then the temperature of alkanethiol adsorption typically ranges from about 20° to about 60° C., from about 22° C. to about 38° C., from about 22° C. to about 28° C., or from about 22° C. to about 26° C. A temperature of at or about 25° C. is particularly suitable for alkanethiol adsorption.

After the alkanethiol has been adsorbed onto the metallized surface of the support, the surface of the support is typically rinsed with ethanol. The ethanol is then usually removed by blowing a stream of N₂ or other inert gas over the rinsed surface.

A DNA hybridization surface for use in a liquid crystal device for determining the presence of a complementary strand of DNA or a DNA fragment in a sample includes a strand of recognition DNA or a DNA recognition fragment which is deposited on a side of the support that contains a surface that preferably drives uniform anchoring of liquid crystals in the absence of the complementary strand of target DNA or complementary target DNA fragment. As noted above, however, all that is required is that the interaction of the complementary strand of DNA or a DNA fragment in a sample with the strand of recognition DNA or a DNA recognition fragment on the DNA hybridization surface results in a visually detectable change in the orientation of a liquid crystal subsequently deposited on the surface. The strand of the recognition DNA or DNA recognition fragment is preferably chemically immobilized on a metallized surface of the support as described above. Although the strand of DNA or DNA fragment may be attached to the surface using various chemical reactions and functional groups known to those skilled in the art, preferably, the strand of DNA or DNA fragment is chemically immobilized on the surface of the support by reaction of a thiol (—SH) group on the DNA recognition fragment thereof with the metal, preferably gold, deposited on the surface of the support. Those skilled in the art will immediately recognize that groups such as phosphines, disulfides, selenols, and other groups which readily bind to metal surfaces may be used in place of the thiol group. Furthermore, those skilled in the art will immediately recognize that intermediary groups may be used to connect the thiol or other functional group to the sequence of nucleic acids in preparing the DNA hybridization surface. The number of nucleic acids in the DNA recognition fragment bound to the support can vary. However, the number of nucleic acids in the strand should be sufficient to provide specific binding of the complementary strand of target DNA that a sample is being tested for. Generally, the number of nucleic acids in the DNA recognition fragment ranges from 5 to 300, from 7 to 100, from 10 to 40, from 12 to 30, and from 15 to 25.

Suitable strands of nucleic acids useful in the present methods may be synthetically produced or derived from DNA, RNA, mRNA (messenger), tRNA (transfer), rRNA (ribosomal), snRNA (small nuclear), snoRNA (small nucleolar), scRNA, hnRNA (heteronuclear), and nucleic acid mimics, such as peptide nucleic acid (PNA) which replaces the nucleic acid sugar-phosphate backbone with a pseudopeptide backbone. The nucleic acid can either be functional, such as a gene, promoter, terminator, or the like, or nonfunctional, as desired. The present invention can be used with nucleic acids whose sequences are undetermined, but are subsequently determined by interaction with the protein or by conventional techniques, such as using nucleic acid probes or sequencing analysis. The nucleic acid can be isolated from a particular source, synthesized or amplified as desired.

Various stringency conditions may be used during the incubation of the DNA hybridization surface and the possible complementary strand of nucleic acids. The terms, high stringency, medium stringency, low stringency and the like encompass meanings well known to those in the art. Generally, “highly stringent conditions” describes conditions which require a high degree of matching to properly hybridize nucleic acids, which typically occurs under conditions of low ionic strength and high temperature. The expression “hybridize under low stringency” commonly refers to hybridization conditions having high ionic strength and lower temperature.

Variables affecting stringency include, for example, temperature, salt concentration, probe/sample homology, nucleic acid length and wash conditions. Stringency is increased with an increase in hybridization temperature, all other factors being equal. Increased stringency provides reduced non-specific hybridization. i.e., less background noise. “High stringency conditions” and “moderate stringency conditions” for nucleic acid hybridizations are explained in Current Protocols in Molecular Biology, Ausubel et al., 1998, Green Publishing Associates and Wiley Interscience, NY, the teachings of which are hereby incorporated by reference. Of course, the artisan will appreciate that the stringency of the hybridization conditions can be varied as desired, in order to include or exclude varying degrees of complementation between nucleic acid strands, in order to achieve the required scope of detection.

The concentration of the DNA recognition fragment in the solution used for DNA fragment adsorption is not critical in the preparation of DNA hybridization surfaces so long as a surface with a suitable thickness of DNA recognition fragment is prepared. DNA recognition fragment adsorption solutions containing the DNA recognition fragments at concentrations ranging from about 1 μM to about 0.1 μM have been employed to prepare suitable DNA hybridization surfaces. Concentrations outside these ranges will also produce suitable surfaces for detecting DNA hybridization. Preferred concentrations range from 0.1 nM to 10 mM and from about 0.01 μM to about 10 mM. The preferred immersion times range from 10 seconds to 24 hours. When using 0.1 nM solutions, the preferred adsorption time ranges from 1 minute to 24 hours with particularly preferred adsorption times ranging from 30 minutes to 15 hours. When using 10 mM solutions, the preferred adsorption times range from 10 seconds to 24 hours, with particularly preferred adsorption times ranging from 30 seconds to 6 hours. A more preferred concentration range is 0.1 μM to 1 μM with preferred immersion times ranging from 10 seconds to 4 hours with an even more preferred concentration being about 0.5 mM with immersion times ranging from 30 seconds to 2 hours.

Generally, the lower the concentration of the DNA recognition fragment in the adsorption solution, the longer the metallized surface will be contacted with the solution. Conversely, the higher the concentration of the DNA recognition fragment in the adsorption solution, the shorter the metallized surface will be contacted with the adsorption solution. One skilled in the art will recognize that lower concentrations of DNA recognition fragment may be used to fine tune the amount of DNA fragment adsorbed onto the metallized surface of the support although longer contact times may be necessary to obtain surfaces with sufficient quantities of adsorbed DNA recognition fragments. Typically, however, the metallized surface is contacted with the DNA recognition fragment adsorption solution for a period of time ranging from about 10 minutes to about 3 hours or from about 30 minutes to about 2 hours.

The adsorption solution containing the DNA recognition fragment is typically an aqueous solution. The aqueous solution is generally buffered with buffers such as, but not limited to, phosphates, Tris, citrates, bicarbonates, and HEPES (4-(2-hydroxyethyl)-1-piperazineethanesulfonic acid). Any buffer suitable for use with DNA or DNA fragments may be used to manufacture a suitable DNA hybridization surface. One particularly useful buffer is KH₂PO₄. The concentration of the buffer in the adsorption solutions may vary considerably. Typically, however, the concentration of a buffer such as KH₂PO₄ in aqueous DNA recognition fragment adsorption solutions ranges from about 0.01 M to about 1 M. The preferred concentrations of salts in the buffer solutions are 50 mM to 1 M with a more preferred range being 100 mM to 1 M. The pH of the adsorption solution containing the DNA recognition fragment typically ranges from 4 to 9, from 6 to 8, from about 6.5 to 7.5, from 6.8 to about 7.2, or about 7.

The DNA recognition fragments are typically adsorbed on the metallized surface of the support in solutions at a temperature ranging from about 15° C. to about 60° C., from about 20° C. to about 40° C., from about 22° C. to about 40° C., or from about 25° C. to about 37° C. Another suitable temperature range is from about 22° C. to about 28° C., and an example of one suitable temperature is about 25° C. As with alkanethiol adsorption, it is not necessary that a steady temperature be maintained during adsorption of the DNA recognition fragment, and temperatures may be increased or decreased during adsorption.

The thickness of DNA recognition fragments on suitable DNA hybridization surfaces may vary considerably. Thicknesses of the DNA recognition fragment on the metallized surface of the support may be measured using various methods including ellipsometry (optical thickness), and x-ray photoelectron spectroscopy. X-ray photoelectron spectroscopy attenuated to gold (4f) or nitrogen (1s) is particularly useful in determining the thickness and relative amount of adsorbed DNA fragments. When attenuated to gold, the thickness of the surface is determined by measuring the decrease in intensity due to interference by the DNA recognition fragment overlying the gold surface. When measuring the intensity of the nitrogen (1s) peak, the relative amount of DNA bound to the metallized surface can be determined making this and especially suitable method for specific quantification of DNA. Generally, thicknesses of the DNA recognition fragment and alkanethiol bound to the metallized surface range from 5 Å (0.5 nm) or about 5 Å (0.5 nm) to 300 Å (30 nm) or about 300 Å (30 nm), from 10 Å (1 nm) or about 10 Å (1 nm) to 100 Å (10 nm) or about 100 Å (10 nm), and from 15 Å (1.5 nm) or about 15 Å (1.5 nm) to 50 Å (5 nm) or about 50 Å (5 nm) as measured by ellipsometry. One example of a suitable thickness is about 30 Å (3 nm).

The DNA hybridization surfaces containing the DNA recognition fragment/alkanethiol mixed monolayers are preferably rinsed prior to use in detecting the presence of a possible complementary DNA fragment or complementary strand of nucleic acids in a sample. Various rinsing conditions have been found to produce suitable DNA hybridization surfaces. The rinsing conditions are employed to remove salts and other materials from DNA hybridization surfaces that might interfere with the interaction of the liquid crystal with the surface. Examples of suitable rinsing conditions include: (1) rinsing the surface with a phosphate buffered aqueous solution followed by rinsing with deionized water; (2) rinsing the surface with a phosphate buffered aqueous solution followed by rinsing with an alcohol such as, but not limited to, ethanol; (3) rinsing the surface with an alcohol, such as, but not limited to, ethanol; and (4) rinsing the surface with distilled or preferably deionized water. In some embodiments, the DNA hybridization surface is rinsed with at least two rinsing solutions. In some such embodiments, the ionic strength of the second solution used to rinse the DNA hybridization surface is lower than the ionic strength of the first solution used to rinse the DNA hybridization surface. In some such methods, at least one of the two rinsing solutions is a phosphate buffered aqueous solution, a Tris buffered aqueous solution, or a sodium chloride solution that includes phosphate or Tris, and at least one of the two rinsing solutions is water, an alcohol, or a combination of water and an alcohol

According to one procedure, a first strand of recognition DNA or DNA recognition fragment with a thiol attached to it, more preferably at one end of the strand or fragment, is delivered to a specific portion of a metallized surface of a support upon which a thiol has been immobilized. In this manner, the first DNA recognition fragment is confined to only a localized area of the surface. A second drop of liquid containing a DNA recognition fragment different from the first DNA fragment is then placed at a second location on the metallized surface of the support. This procedure is repeated until the metallized surface of the support includes an array of areas, each of which is covered by different DNA recognition fragments. This procedure provides a surface suitable for use in analyzing samples that may contain more than one complementary strand of DNA or fragment thereof. Those skilled in the art will recognize that variations on the above procedure could also be used to produce a multiarray. In one such procedure, rather than “spotting” droplets of liquid on a surface, a fluidic channel (e.g., made from micromolded polydimethylsiloxane) is used to deliver liquids to localized regions of a surface. In another such procedure, microcontact printing is used to deliver the reagents to the surface. Generally, any method known to those skilled in the art for delivering liquids to localized regions of a surface could be used to produce the preferred microarray devices for detecting multiple target DNA fragments.

In one alternative embodiment of the procedure described in the above paragraph, the same DNA recognition fragment is placed on various distinct portions of a surface to create a surface with multiple detection areas that may be used to analyze several or numerous samples for the presence of a strand of DNA or DNA fragment or strand of nucleic acids complementary to that deposited on the surface.

The microarrays presented above provide a device for detecting the presence of more than one complementary strand of DNA or fragment thereof in a sample. The device includes a support, preferably with obliquely deposited gold over titanium as described above. The device also includes a first DNA or DNA fragment detection region on a first portion of the support. The first DNA or DNA fragment detection region includes a first DNA recognition fragment thereof bound to the surface which recognizes and binds a first complementary strand of DNA or fragment thereof in a sample. The device further includes at least one other DNA or DNA fragment detection region on at least one other portion of the support, and the at least one other DNA or DNA fragment detection region includes at least one other different DNA recognition fragment thereof bound to the surface which recognizes and binds a second complementary strand of DNA or fragment thereof in a sample. The first DNA or DNA fragment detection region preferably uniformly anchors liquid in the absence of the first complementary strand of DNA or fragment thereof in a sample, and the at least one other DNA or DNA fragment detection region preferably uniformily anchors liquid crystals in the absence of the at least one other strand of complementary strand of DNA or fragment thereof. The uniform anchoring of liquid crystals in the first DNA or DNA fragment detection region is disrupted when the first DNA or DNA fragment detection region is exposed to the first complementary strand of DNA or fragment thereof, and the uniform anchoring of liquid crystals in the at least one other target species detection region is disrupted when the at least one other target species detection region is exposed to the at least one other target species.

The DNA hybridization surface of the present invention allows for detection of complementary strands or fragments of DNA in dilute solutions. No fluorescent or other labeling is required. This would not be possible using lyotropic liquid crystals due to the presence of the diluting solvent required in the preparation of lyotropic liquid crystals unless the DNA recognition fragment is immobilized on the surface as in the DNA hybridization surfaces of the present invention. Thus, the DNA hybridization surfaces of the present invention with surface-immobilized DNA recognition fragments may be used in conjunction with lyotropic liquid crystals. Additionally, in the DNA hybridization surfaces of the present invention, any interaction between the DNA recognition fragment bound to the metallized surface and a complementary or non-complementary DNA fragment in a solution to be tested occurs before the liquid crystal contacts the DNA hybridization surface. Therefore, the DNA hybridization surfaces avoid undesirable interactions between liquid crystals and DNA fragments. An additional advantage of the DNA hybridization surfaces of the present invention is that patterned surfaces may be readily prepared as described above to produce microarray and multiarray devices.

After the DNA hybridization surface has been prepared and preferably rinsed as described above, the surface is ready for use in detecting DNA hybridization. A fragment of potentially complementary DNA is obtained and isolated from samples such as dried blood drops using known procedures. Aqueous solutions containing the possibly complementary DNA strand or fragments of strands are then prepared using procedures known to those skilled in the art. Such aqueous solutions preferably contain the potentially complementary DNA at concentrations ranging from about 0.1 μM to 1.0 μM, from about 0.3 μM to about 0.8 μM, from about 0.5 μM to about 1.0 μM, from about 0.1 μM to about 0.6 μM, or about 0.5 μM although the concentration of the complementary DNA in the sample will be dictated by the sample. Although the above ranges are preferred, the concentration of the complementary DNA in the sample may range considerably such as from sub-picomolar to millimolar. Such aqueous solutions are then contacted with the DNA hybridization surface for an incubation time ranging from about 1 to about 24 hours, or from about 3 to about 24 hours. Incubation time and concentration may vary. Typically, the lower the concentration, the longer the incubation time should be. Therefore, the concentration of the aqueous solution containing the possibly complementary strand of DNA and the incubation time should be adjusted such that a sufficient amount of DNA hybridization will occur if the complementary DNA fragment is present and result in subsequent disruption of the uniform anchoring of liquid crystal.

The temperature at which aqueous solutions containing the possibly complementary strands of DNA are incubated with the DNA hybridization surface may vary considerably. Preferred temperatures range from about 18° C. to about 60° C., from about 20° C. to about 40° C., from about 22° C. to about 37° C., from about 22° C. to about 28° C., and from about 22° C. to about 26° C. An incubation temperature of about 25° C. has been found to be particularly suitable.

The aqueous incubation solution possibly containing the complementary strand or fragment of DNA typically contains a buffer suitable for use with DNA and DNA fragments. Examples of suitable buffer solutions include, but are not limited to, the following: (1) aqueous solutions containing Tris at a concentration of about 10 mM; EDTA at a concentration of about 1 mM; and sodium chloride at a concentration ranging from about 0.1 to about 1.0 M; (2) aqueous solutions containing Tris at a concentration of about 10 mM and sodium chloride at a concentration ranging from about 0.1 to about 1.0 M; (3) aqueous solutions containing Tris at a concentration of about 10 mM; and (4) aqueous solutions containing sodium or potassium phosphate at a concentration of about 0.01 to about 1.0 M. Those skilled in the art will recognize that other buffer systems will be suitable for use in the present invention.

After the DNA hybridization surface has been contacted with the aqueous solution containing a possibly complementary strand or fragment of DNA for a suitable time, the resulting DNA hybridization surface is preferably rinsed to produce a rinsed incubated DNA hybridization surface that is preferably substantially free of excess sodium salts, potassium salts, and Tris salts. Proper rinsing of the incubated DNA hybridization surface has been found to improve performance in detecting DNA hybridization. Various solutions may be used to rinse the DNA hybridization surface after incubation. Examples of suitable rinsing solutions and conditions include, but are not limited to: (1) distilled or deionized water; (2) ethanol; (3) deionized water and then an alcohol such as ethanol; (4) a solution of the incubation buffer solution without the possibly complementary strand of DNA and then deionized or distilled water; (5) a solution of the incubation buffer solution without the possibly complementary strand of DNA and then an alcohol such as ethanol; and (6) a solution of the incubation buffer solution without the possibly complementary strand of DNA; then deionized or distilled water; and then an alcohol such as ethanol. Generally, it will be noted that the final solution used to rinse the incubated DNA hybridization surface will be one free of salts or will be a solution with a low concentration of salts such that the orientation of the liquid crystal is not perturbed by the presence of salts remaining on the surface after rinsing with the final solution. The rinsing conditions described in (5) above have been found to be particularly suitable for rinsing incubated hybridization surfaces.

Various types of liquid crystals may be used in conjunction with the present invention. Examples of these include both nematic and smectic liquid crystals. Other classes of liquid crystals that may be used in accordance with the invention include, but are not limited to: polymeric liquid crystals, thermotropic liquid crystals, lyotropic liquid crystals, columnar liquid crystals, nematic discotic liquid crystals, calamitic nematic liquid crystals, ferroelectric liquid crystals, discoid liquid crystals, and cholesteric liquid crystals. Examples of just some of the liquid crystals that may be used are shown in Table 1. A particularly preferred liquid crystal for use in the present invention includes 4-cyano-4′-pentylbiphenyl (5CB).

TABLE 1
Molecular Structure of Mesogens Suitable for use in Detecting DNA Hybridization.

| Mesogen | Structure |
|---|---|
| Anisaldazine | 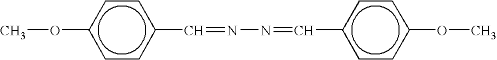 |
| NCB | 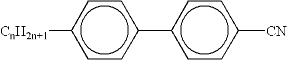 |
| CBOOA | 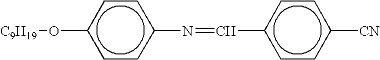 |
| Comp A | 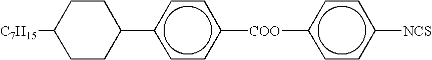 |
| Comp B | 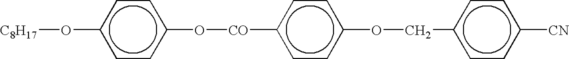 |
| DB₇NO₂ | 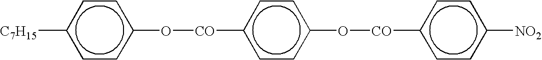 |
| DOBAMBC | 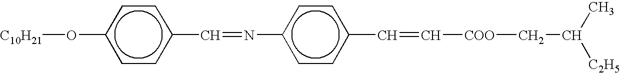 |
| nOmn = 1, m = 4: MBBAn = 2, m = 4: EBBA | 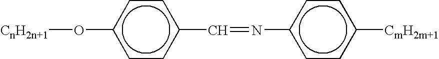 |
| nOBAn = 8: OOBAn = 9: NOBA | 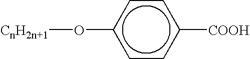 |
| nmOBC | 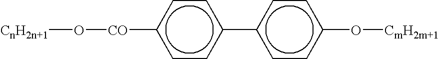 |
| nOCB | 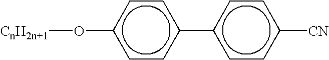 |
| nOSI | 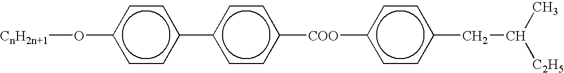 |
| 98P | 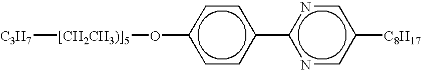 |
| PAA | 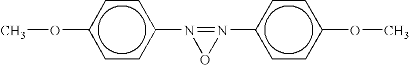 |
| PYP906 | 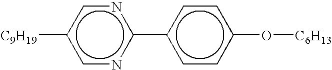 |
| n̅Sm | 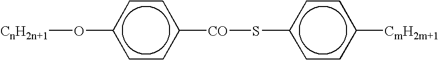 |

An optical cell for use in detecting DNA hybridization preferably includes a DNA hybridization surface as described above. An optical cell may also include a spacing material, preferably a film, positioned parallel to but a spaced distance away from the top surface of the DNA hybridization surface. The spacing material and the top surface of the DNA hybridization surface thus define a cavity that may be filled with a liquid crystal. An optical cell may also contain another surface that uniformly anchors liquid crystals positioned parallel to and over the top of the DNA hybridization surface. Typically, the spacing material such as a film is positioned between the DNA hybridization surface and the surface that uniformly anchors liquid crystals. It is not required that both surfaces of the optical cell be DNA hybridization surfaces. The spacing material is preferably a film of a defined thickness that is preferably stable in the presence of the liquid crystal material, easy to handle, and does not contaminate the liquid crystal. A variety of films may be suitable for use as spacing materials in the optical cells according to the invention as will be apparent to those skilled in the art. A preferred film spacing material is preferably made of a polymeric material such as Mylar® brand film or Saran® brand wrap. The film spacing material is typically placed between the top surface of the DNA hybridization surface and the surface that uniformly anchors liquid crystals such that the top surface of the DNA hybridization surface and the surface that uniformly anchors liquid crystals face each other. The spacing material may also be comprised of rods or microparticles such as microspheres of defined diameter that are dispersed into the liquid crystal so as to separate the two surfaces forming the optical cell.

After the DNA hybridization surface has been contacted for a suitable time with a target solution potentially containing a complementary strand or fragment of DNA and the DNA hybridization surface has preferably been rinsed, a liquid crystal is drawn into the area between the DNA hybridization surface and the surface that uniformly anchors liquid crystals in the optical cell. Various materials may be used as the surface that uniformly anchors liquid crystals in the optical cells including, but not limited to rubbed surfaces, glass surfaces modified by reaction with octadecyltrichlorosilane and glass surfaces with obliquely deposited gold films. Other suitable surfaces that uniformly anchor liquid crystals include rubbed glass slides and glass slides with shear-deposited Teflon. As long as the surface uniformly anchors liquid crystals, the presence of a target complementary DNA strand in a sample will disrupt the anchoring of the liquid crystal on the DNA hybridization surface and will thus be detected due to the disruption in the anchoring of the liquid crystal on the DNA hybridization surface.

Preferred kits for use in detecting hybridization of DNA on a surface typically include a metallized surface according to the invention; a liquid crystal; a surface that uniformly anchors liquid crystals; and a spacing material such as a film adapted to be placed between the DNA hybridization surface and the surface that uniformly anchors liquid crystals such that an optical cell, as described above, may be manufactured. Any of the kits of the present invention preferably provide either an alkanethiol or a metallized surface to which a suitable alkanethiol has already been adsorbed. If the alkanethiol is provided separately, then it may be in the form of a solution such as an ethanolic solution or in a form for addition to a liquid to prepare an alkanethiol solution for adsorption to the metallized surface. The surface that uniformly anchors liquid crystal provided in preferred kits may include any of those described above. Suitable kits of the invention may also include one or more rinsing solution(s) for use after adsorption of an alkanethiol and a thiolated DNA fragment, and after incubation with a sample solution. Such kits may include instructions for the detection of DNA hybridization and/or instructions for assembling a DNA hybridization surface or an optical cell for detecting the presence of a complementary strand of DNA. Such instructions will typically include directions for incubating the DNA hybridization surface with a sample that possibly contains a strand of DNA or fragment of a strand of DNA that is complementary to the DNA recognition fragment bound to the metallized surface of the support in the DNA hybridization surface. Such a kit will also contain a description of conditions for adsorbing the DNA recognition fragment to the metallized surface and for rinsing the DNA hybridization surface. It will also preferably contain instructions explaining how the presence of a complementary strand of DNA is identified and may also contain steps that may be used to determine the concentration of the complementary DNA strand in a sample.

Other kits according to the present invention include at least one metallized surface and a liquid crystal. Such kits will preferably contain a metallized surface that comprises an adsorbed alkanethiol or the alkanethiol as described in the preceding paragraph. These kits may also be used to detect the presence of a strand of complementary DNA in a sample. The method for detecting the complementary DNA strand with such a kit includes forming a DNA hybridization surface using the metallized surface, an alkanethiol if it is not already adsorbed onto the metallized surface, and a DNA recognition fragment containing a functional group for adsorption to the metallized surface, rinsing the DNA hybridization surface, and contacting a portion of the DNA hybridization surface with a quantity of the sample; placing the liquid crystal of the kit on the portion of the DNA hybridization surface that contacted the sample; and determining whether the uniform anchoring of the liquid crystal has been disrupted. If the uniform anchoring of the liquid crystal has been disrupted, then the complementary strand of DNA is present in the sample. Determining whether the uniform anchoring of the liquid crystal has been disrupted may be accomplished by various methods. One such method includes viewing the DNA hybridization surface with the liquid crystal on it through cross polarizers.

A method for detecting the presence of a complementary strand of DNA with an optical cell includes several steps. First, a DNA hybridization surface is incubated with a sample to be tested for the complementary strand of DNA. Typically, the incubation period will range from 1 to 24 hours, but this will vary depending on the suspected or known concentration of DNA strands or fragments in the sample. Second, a spacing material such as a film is placed between the incubated DNA hybridization surface and the surface that uniformly anchors liquid crystals such that the top surface of the DNA hybridization surface faces the surface that uniformly anchors liquid crystals. Third, a liquid crystal such as 5CB is drawn into the area between the incubated DNA hybridization surface and the surface that uniformly anchors liquid crystals. Typically, the liquid crystal is in an isotropic phase during this step. The liquid crystal may need to be heated prior to drawing it into the area between the incubated DNA hybridization surface and the surface that uniformly anchors the liquid crystal. The liquid crystal can also be drawn into the cell in the nematic phase. Finally, the person conducting the assay determines whether the liquid crystal is uniformly anchored on the rubbed substrate structure by the methods described herein. If the liquid crystals are uniformly anchored on the DNA hybridization surface, the sample will be found to not contain the complementary strand of DNA. On the other hand, if the liquid crystal is not uniformly anchored on the incubated DNA hybridization surface, then the sample will be found to contain the complementary strand of DNA.

In addition to the method described above, kits and optical cells to be used in accordance with the present invention may also be designed such that the sample to be tested is passed directly through or maintained in a preassembled cell including the DNA hybridization surface, the spacing material, and the surface that uniformly anchors the liquid crystals. Once a sufficient time has passed, the sample may be removed followed by addition of liquid crystal to determine whether or not the complementary strand of DNA was present in the sample.

In addition to the methods described above, kits and devices may also be designed such that liquid crystal is placed directly onto the surface of an incubated DNA hybridization surface and the orientation of the liquid crystal is observed with one surface of the liquid crystal on the DNA hybridization surface being a surface with air. That is, the liquid crystal is simply placed onto the top of the DNA hybridization surface. It is well known that the orientation of 5CB, for example, is homeotropic at the liquid-crystal air interface. Thus, the free surface of the liquid crystal can substitute for the second surface that uniformly anchors the liquid crystal. This type of kit is particularly useful for microarrays of patterned recognition groups.

### EXAMPLES

The following materials and methodologies were utilized in the examples discussed in greater detail below.

### Materials

Glass microscope slides used in the experiments were marked premium grade and obtained from Fisher Scientific (Pittsburgh, Pa.). Glass slides were cleaned prior to use by treating with “piranha solution” (70% H₂SO₄/30% H₂O₂). “Piranha solution” should be handled with extreme caution because it reacts violently with organic materials and should not be stored in closed containers. After cleaning for 1 hour at 80° C. in “piranha solution”, the slides were rinsed copiously in deionized water, and dried under a stream of nitrogen. Prior to use, the clean substrates were stored in an oven heated at 120° C. for at least 3 hours.

Hexanethiol was obtained from Sigma (St. Louis, Mo.). Both the thiol-derivatized DNA recognition fragment and the complementary DNA fragment were synthesized on an ABI DNA synthesizer (Applied Biosystems, Inc., Foster City, Calif.). The DNA recognition fragment was modified at the 5′ terminus by a 5′-thiol-modified C₆ (Glen Research). The precursors for the 4 common DNA bases (A, C, T, G) and the thiol modifier were purchased from Glen Research. The reagents were diluted in acetonitrile and placed into the DNA synthesizer model 394 (Applied Biosystems Inc.). The synthesis of the oligonucleotides was accomplished by sequential, automated addition of the bases from the 3′ base to the 5′ base. The thiol modifier was added in an identical fashion to the final 5′ base. After completion of the synthesis, the single-stranded DNA was deprotected and lyophilized. The fragments were subsequently purified by reverse-phase binary gradient elution HPLC (Shimadzu SCL-10AVP) prior to use. The concentration of DNA in the purified solution was measured with an HP8452A UV-visible spectrophotometer. Buffer solutions were prepared using analytical grade commercially available reagents. The nematic liquid crystal, 4-cyano-4′-pentylbiphenyl (5CB), manufactured by BDH was purchased from EM industries (Hawthorne, N.Y.).

### Optical Cells

Optical cells were prepared by pairing two glass slides and by spacing one side of them apart using ˜10 μm thick films of Mylar® brand film obtained from Dupont Films (Wilmington, Del.). One of the slides was a DNA hybridization surface according to the invention, and the other was typically a second DNA hybridization surface of the same preparation. Additional substrates could be used as the second surface including, but not limited to, metallic or glass substrates chemically modified to host a variety of molecules such as alkanethiols and silanes. The cells were held together using “bulldog” clips placed along the edge of the glass microscope slides. The cell was placed on a hot plate at 40° C. and heated with hot air for approximately 10 seconds. The nematic liquid crystal of 5CB was heated into its isotropic phase (˜35° C.) in a glass syringe. A drop of 5CB was then placed on the edge of each cell on the hot plate. The 5CB was then drawn into the optical cells by capillary action. Once the optical cells were filled with 5CB, the cell was removed from the hot plate and cooled in air to room temperature. Upon cooling, the isotropic phase of 5CB transformed to the nematic phase.

### Polarized Light Microscopy

A polarized light microscope (BX60, Olympus, Tokyo, Japan) was used to observe the optical textures formed by light transmitted through the optical cells filled with 5CB. All images were obtained using a 10× objective lens with a 1 mm field of view between cross-polars. Images of the optical appearance of liquid crystal optical cells prepared from the DNA hybridization surfaces were captured with a digital camera (C-2020 Z, obtained from Olympus America Inc. (Melville, N.Y.)) that was attached to the polarized light microscope. The pictures were obtained using high quality mode (resolution of 1600×1200 pixels) at an aperture of f11 and shutter speed of 1/160 seconds.

### Ellipsometric Thickness

The sample substrates for measurement were prepared using the same procedure used to prepare the glass slides for optical measurement. Ellipsometric thickness was measured at three points on each sample using a Rudolph Auto EL ellipsometer (Flanders, N.J.) at a wavelength of 6320 Å (632 nm) and an angle of incidence of 70°. The ellipsometer used the Null method to obtain Ψ and Δ directly. In order to interpret the ellipsometric thickness of bound DNA recognition fragment, the Ψ and Δ obtained from the ellipsometer were used in a combination of the Fresnel equations and Snell's law. To perform the calculations, a refractive index of 1.46 was used for the organic films formed on the gold-coated glass slides, and the gold layer was assumed to be semi-infinite reducing the calculations to only the gold substrate layer and the adsorbed DNA and alkanethiol layer.

### X-Ray Photoelectron Spectroscopy (XPS) Thickness Measurements

For XPS measurements (Surface Science, Mountain View, Calif.), the same glass slides used for the ellipsometric measurements were used. The x-ray source was an Al Kα anode with a spot size of 250 mm×1000 mm. During the XPS measurements, the chamber pressure remained <1×10⁻⁸ torr (typically 2×10⁻⁹ torr). At each point sampled on the surface, a survey scan was conducted to obtain the binding energy profile of all emitted electrons. Specific acquisition of elemental peaks was performed in Resolution 4 with 50 scans centered on the Au (4f), N (1s), C (1s), O (1s), and Na (1s) peaks. Attenuation of the intensity of the Au (4f_{7/2}) peak was used to estimate the thickness of adsorbed layers on the glass slides. Specifically, the intensity of the Au (4f_{7/2}) peak was plotted against the ellipsometric thickness of adsorbed layers of alkanethiols (C₄-C₂₀) to obtain a standard curve. The measured intensity of the Au (4f_{7/2}) peak of the DNA and alkanethiol samples was then compared against this curve. The relative heights of the N (1s) peaks (sample to sample comparison) was used to quantify the amount of bound DNA on the surface—in contrast, no N (1s) peak was observed for pure alkanethiol layers. Measurements of ratios of C (1s) and O (1s) peaks were used throughout the surveys to check for oxidation of the surface by the x-ray source. Typically, no oxidation (signified by an increase in the oxygen to carbon ratio) was observed to occur during the measurements. Finally, the Na (1s) peak was followed to assess the degree of salt precipitation on the surfaces due to improper rinsing of the samples (sodium chloride at 1 M was contained in the hybridization buffer). Typically, no, or minimal, signal was observed at the Na (1s) peak.

### Discussion of Experimental Results

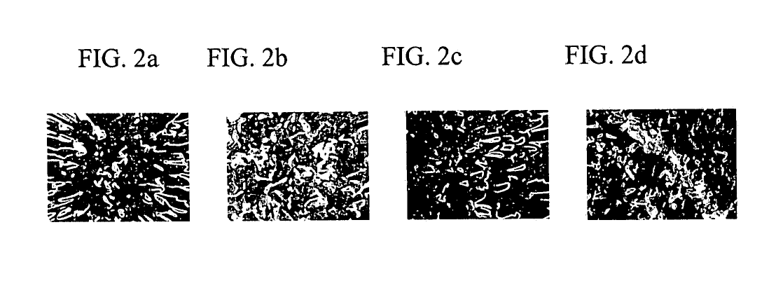

FIGS. 2a-2d are scanned images of the optical textures of optical cells taken through a polarized light microscope and prepared with 5CB. Each of the glass slides used to prepare the optical cells contained a surface with obliquely deposited gold on it which was immersed in an ethanolic solution of 1 mM hexanethiol for 60 minutes at 37° C. and was then immersed for 0.5 hours (FIG. 2a), 1.5 hours (FIG. 2b), 2.5 hours (FIG. 2c), and 24 hours (FIG. 2d) at 37° C. in a 1 mM aqueous KH₂PO₄ buffered solution containing 5′-HS—(CH₂)₆-TGC-AGT-TCC-GGT-GGC-TGA-TC-3′ at a concentration of 0.5 μM.

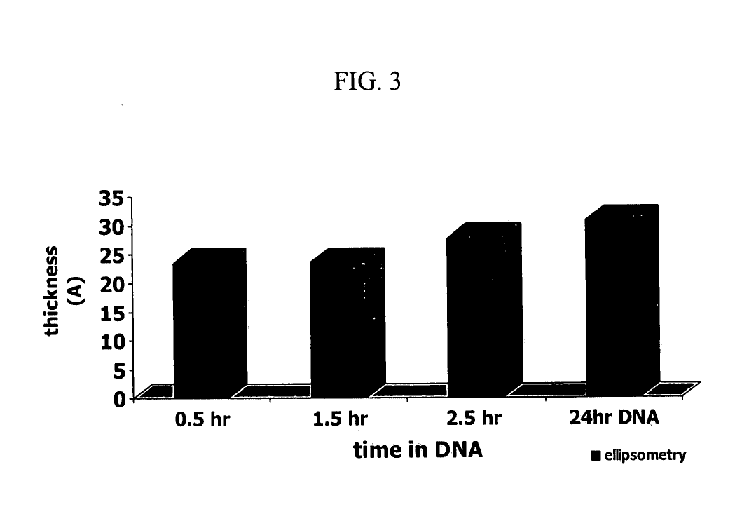

FIG. 3 is a graph showing the ellipsometric thicknesses (Å) of surfaces of glass slides containing obliquely deposited gold after the slides had been immersed in ethanolic solutions of 1 mM hexanethiol for 60 minutes at 37° C. and then for 0.5 hours, 1.5 hours, 2.5 hours, and 24 hours at 37° C. in a 1 M aqueous KH₂PO₄ buffered solutions containing 5′-HS—(CH₂)₆-TGC-AGT-TCC-GGT-GGC-TGA-TC-3′ at a concentration of 0.5 μM.

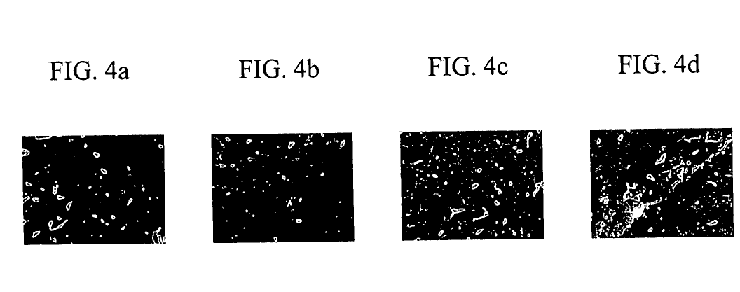

FIGS. 4a-4d are scanned images of the optical textures of optical cells taken through a polarized light microscope and prepared with 5CB. Each of the glass slides used to prepare the optical cells contained a surface with obliquely deposited gold on it which was immersed in an ethanolic solution of 1 mM hexanethiol for 60 minutes at 37° C. and was then immersed for 0.5 hours (FIG. 4a), 1.5 hours (FIG. 4b), 2.5 hours (FIG. 4c), and 24 hours (FIG. 4d) at 25° C. in a 1 M aqueous KH₂PO₄ buffered solutions containing 5′-HS—(CH₂)₆-TGC-AGT-TCC-GGT-GGC-TGA-TC-3′ at a concentration of 0.5 μM.

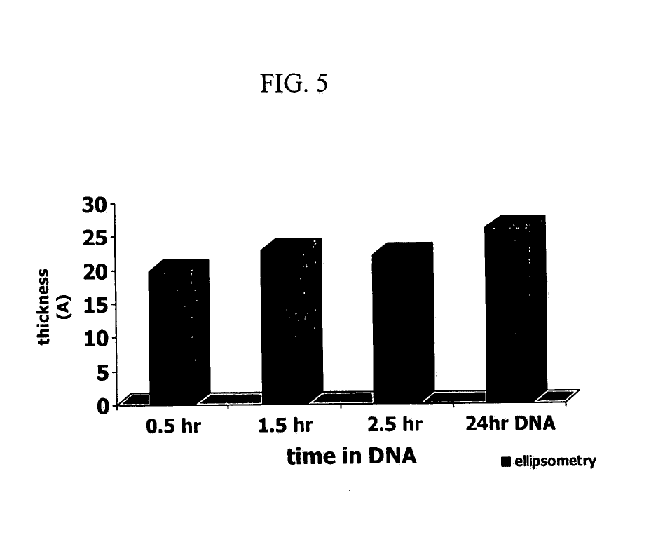

FIG. 5 is a graph showing the ellipsometric thicknesses (Å) of surfaces of glass slides containing obliquely deposited gold after the slides had been immersed in ethanolic solutions of 1 mM hexanethiol for 60 minutes at 37° C. and then for 0.5 hours, 1.5 hours, 2.5 hours, and 24 hours at 25° C. in a 1 M aqueous KH₂PO₄ buffered solutions containing 5′-HS—(CH₂)₆-TGC-AGT-TCC-GGT-GGC-TGA-TC-3′ at a concentration of 0.5 μM.

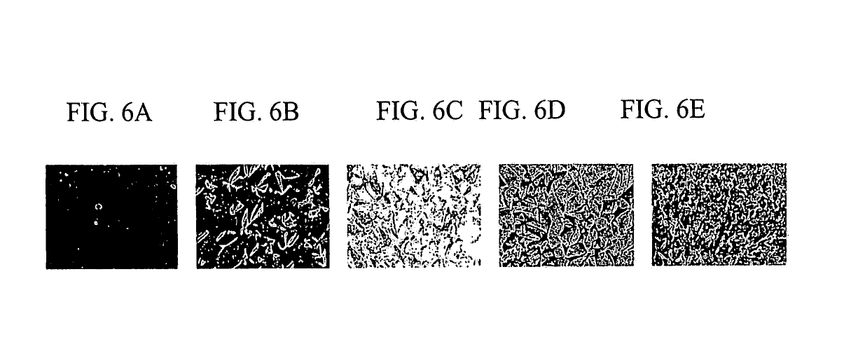

FIGS. 6a-6e are scanned images of the optical textures of optical cells taken through a polarized light microscope and prepared with 5CB. Each of the glass slides used to prepare the optical cells contained a surface with obliquely deposited gold on it which was first immersed for 0.0 minutes (FIG. 6a), 0.5 minutes (FIG. 6b), 3 minutes (FIG. 6c), 5 minutes (FIG. 6d), and 48 hours (FIG. 6e) at 25° C. in a 1 M aqueous KH₂PO₄ buffered solutions containing 5′-HS—(CH₂)₆-TGC-AGT-TCC-GGT-GGC-TGA-TC-3′ at a concentration of 1.0 μM and at 25° C. and which was then rinsed with deionized water and then immersed in an ethanolic solution of 1 mM hexanethiol for 60 minutes at 25° C. The glass slide used to prepare the optical cell for FIG. 6e was not immersed in the ethanolic hexanethiol solution.

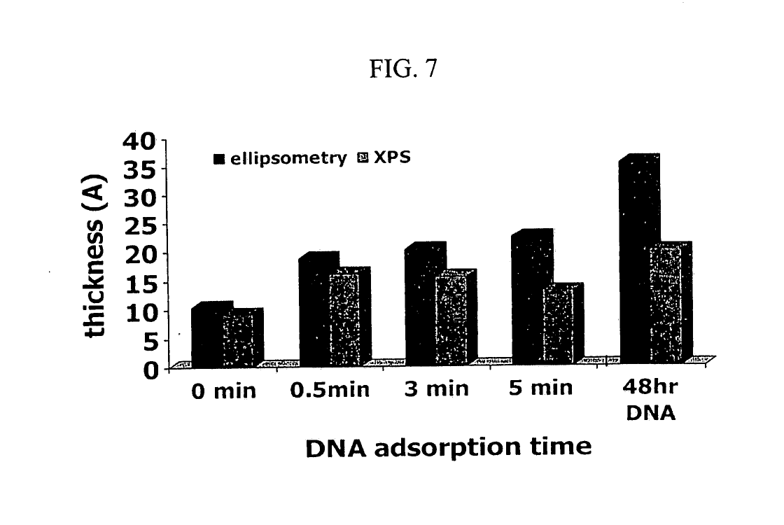

FIG. 7 is a graph showing the thicknesses (Å) measured using ellipsometry (first bar) and x-ray photoelectron spectroscopy (Au) (second bar) of surfaces of glass slides containing obliquely deposited gold after the slides were first immersed for 0.0 minutes, 0.5 minutes, 3 minutes, 5 minutes, and 48 hours at 25° C. in a 1 M aqueous KH₂PO₄ buffered solutions containing 5′-HS—(CH₂)₆-TGC-AGT-TCC-GGT-GGC-TGA-TC-3′ at a concentration of 1.0 μM, were then rinsed with deionized water, and were then immersed in an ethanolic solution of 1 mM hexanethiol for 60 minutes at 25° C. The glass slide immersed in the 5′-HS—(CH₂)₆-TGC-AGT-TCC-GGT-GGC-TGA-TC-3′ for 48 hours was not immersed in the ethanolic hexanethiol solution.

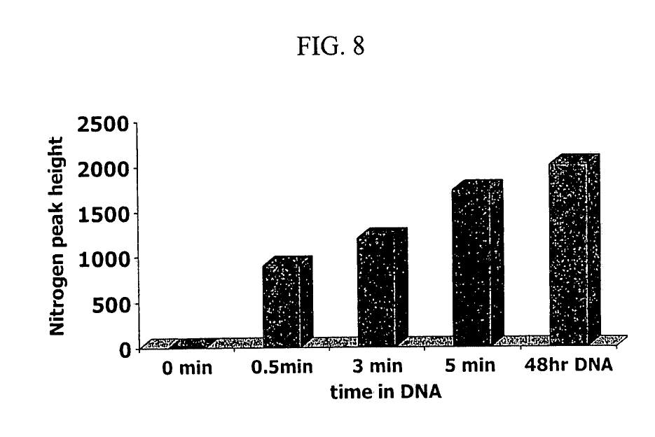

FIG. 8 is a graph showing the nitrogen peak height measured using x-ray photoelectron spectroscopy (N (1s)) of surfaces of glass slides containing obliquely deposited gold after the slides were first immersed for 0.0 minutes, 0.5 minutes, 3 minutes, 5 minutes, and 48 hours at 25° C. in a 1 M aqueous KH₂PO₄ buffered solutions containing 5′-HS—(CH₂)₆-TGC-AGT-TCC-GGT-GGC-TGA-TC-3′ at a concentration of 1.0 μM, were then rinsed with deionized water, and were then immersed in an ethanolic solution of 1 mM hexanethiol for 60 minutes at 25° C. The glass slide immersed in the 5′-HS—(CH₂)₆-TGC-AGT-TCC-GGT-GGC-TGA-TC-3′ for 48 hours was not immersed in the ethanolic hexanethiol solution. FIG. 8 shows that the nitrogen peak heights obtained using x-ray photoelectron spectroscopy attenuated to N correspond very well with the amount of time that a metallized surface is immersed in a DNA fragment adsorption solution.

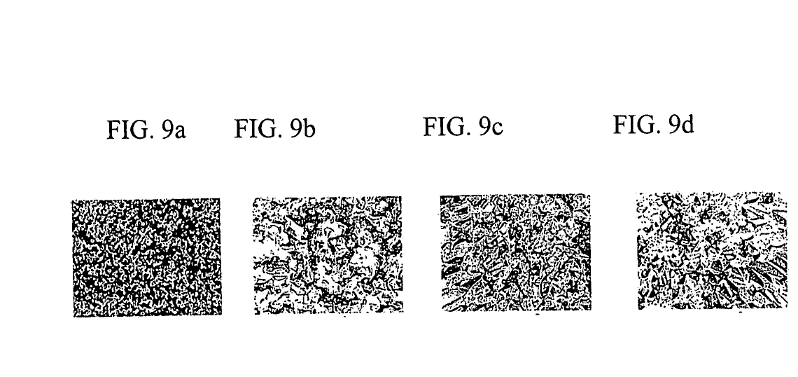

FIGS. 9a-9d are scanned images of the optical textures of optical cells taken through a polarized light microscope and prepared with 5CB. The glass slides used to prepare the optical cells contained a surface with obliquely deposited gold on it which was immersed for 1.0 minute at 25° C. in a 1 M aqueous KH₂PO₄ buffered solution containing 5′-HS—(CH₂)₆-TGC-AGT-TCC-GGT-GGC-TGA-TC-3′ at a concentration of 1.0 μM and was then immersed in an ethanolic solution of 1 mM hexanethiol for 60 minutes at 25° C. The slides were then rinsed with an aqueous solution of TE (FIG. 9a), deionized water (FIG. 9b), with an aqueous solution of TE and then with deionized water (FIG. 9C), or with an aqueous solution of TE and then with ethanol (FIG. 9d). The thicknesses of the DNA fragment and alkanethiol deposited on the metallized surface for each of the slides used were 29.3 Å (2.93 nm) (FIG. 9a); 20.6 Å (2.06 nm) (FIG. 9b); 22.3 Å (2.23 nm) (FIG. 9C); and 15.4 Å (1.54 nm) (FIG. 9d) as measured using ellipsometry. The significant differences in the optical textures shows that rinsing conditions are an important consideration in optimizing performance of DNA hybridization surfaces.

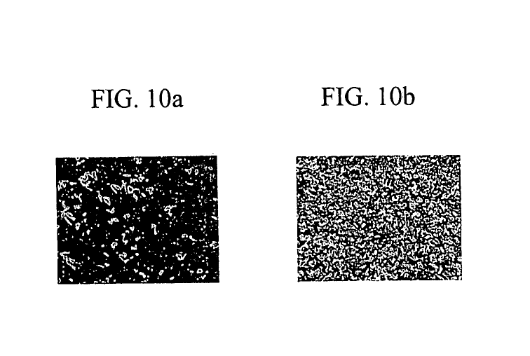

FIGS. 10a and 10b are scanned images of the optical textures of optical cells taken through a polarized light microscope and prepared with 5CB. The glass slides used to prepare the optical cells contained a surface with obliquely deposited gold on it which was immersed for 5.0 minutes at 25° C. in a 1 M aqueous KH₂PO₄ buffered solution containing 5′-HS—(CH₂)₆-TGC-AGT-TCC-GGT-GGC-TGA-TC-3′ at a concentration of 0.5 μM and was then immersed in an ethanolic solution of 1 mM hexanethiol for 60 minutes at 25° C. FIG. 10a is a scanned image of the optical texture of an optical cell made using a slide prepared as above, but after incubation in a aqueous solution of TE at 25° C. for 24 hours. FIG. 10b is a scanned image of the optical texture of an optical cell made using a slide prepared as described above for 9a, but after incubation for 24 hours in an aqueous TE solution containing 5′-GAT-CAG-CCA-CCG-GAA-CTG-CA-3′ at a concentration of 1 μM and a temperature of 25° C. The significant increase in non-uniformity of the liquid crystal that occurred upon incubation in a solution with a complementary target DNA fragment indicates that binding of the complementary DNA fragment disrupts the ability of the DNA hybridization surface to uniformly anchor liquid crystals. The thicknesses of the DNA and alkanethiol on the metallized surface for each of the slides used were 15.7 Å (1.57 nm) (FIG. 10a) and 21.0 Å (2.10 nm) (FIG. 10b) as measured using ellipsometry. The increase in thickness that occurred upon incubation in the solution containing the complementary DNA fragment is further evidence of the hybridization of DNA on the DNA hybridization surface.

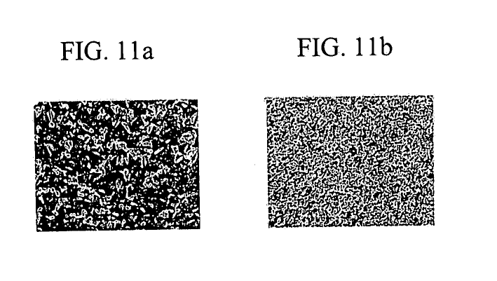

FIGS. 11a and 11b are scanned images of the optical textures of optical cells taken through a polarized light microscope and prepared with 5CB. The glass slides used to prepare the optical cells contained a surface with obliquely deposited gold on it which was immersed for 10.0 minutes at 25° C. in a 1 M aqueous KH₂PO₄ buffered solution containing 5′-HS—(CH₂)₆-TGC-AGT-TCC-GGT-GGC-TGA-TC-3′ at a concentration of 0.5 μM and was then immersed in an ethanolic solution of 1 mM hexanethiol for 60 minutes at 25° C. FIG. 11a is a scanned image of the optical texture of an optical cell made using a slide prepared as above, but after incubation in a aqueous solution of TE at 25° C. for 4 hours. FIG. 11b is a scanned image of the optical texture of an optical cell made using a slide prepared as described above, but after incubation for 4 hours in an aqueous TE solution containing 5′-GAT-CAG-CCA-CCG-GAA-CTG-CA-3′ at a concentration of 1 μM and a temperature of 25° C. The significant increase in non-uniformity of the liquid crystal that occurred upon incubation in a solution with a complementary target DNA fragment indicates that binding of the complementary DNA fragment disrupts the ability of the DNA hybridization surface to uniformly anchor liquid crystals. The thicknesses of the DNA and alkanethiol on the metallized surface for each of the slides used were 17.6 Å (1.76 nm) (FIG. 11a) and 28.1 Å (2.81 nm) (FIG. 11b) as measured using ellipsometry. The increase in thickness that occurred upon incubation in the solution containing the complementary DNA fragment is further evidence of the hybridization of DNA on the DNA hybridization surface.

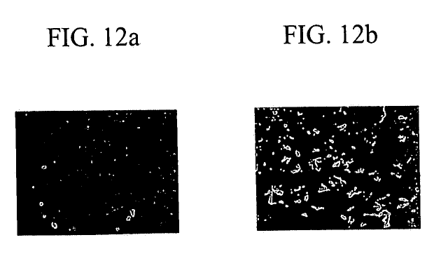

FIGS. 12a and 12b are scanned images of the optical textures of optical cells taken through a polarized light microscope and prepared with 5CB. The glass slides used to prepare the optical cells contained a surface with obliquely deposited gold on it which was immersed for 10 minutes at 25° C. in a 1 M aqueous KH₂PO₄ buffered solution containing 5′-HS—(CH₂)₆-TGC-AGT-TCC-GGT-GGC-TGA-TC-3′ at a concentration of 0.1 μM and was then immersed in an ethanolic solution of 1 mM hexanethiol for 60 minutes at 25° C. FIG. 12a is a scanned image obtained from the slide prepared as described above after overnight immersion in a TE solution, and FIG. 12b is a scanned image of the slide prepared as above after overnight immersion in an aqueous TE buffered solution containing 5′-GAT-CAG-CCA-CCG-GAA-CTG-CA-3′ at a concentration of 1 μM and a temperature of 25° C. and treatment with a liquid crystal. The significant increase in non-uniformity of the liquid crystal that occurred upon incubation in a solution with a complementary target DNA fragment indicates that binding of the complementary DNA fragment disrupts the ability of the DNA hybridization surface to uniformly anchor liquid crystals. The thicknesses of the DNA and alkanethiol on the metallized surface for each of the slides used were 12.6 Å (1.26 nm) (FIG. 12a) and 20.5 Å (2.05 nm) (FIG. 12b) as measured using ellipsometry. The increase in thickness that occurred upon incubation in the solution containing the complementary DNA fragment is further evidence of the hybridization of DNA on the DNA hybridization surface.

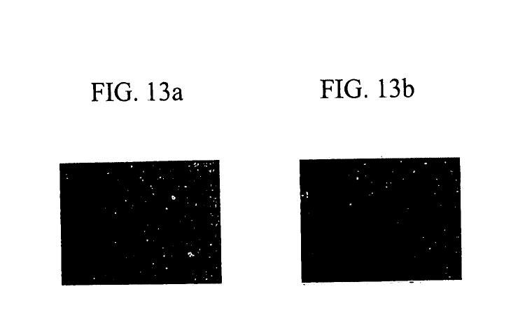

FIGS. 13a and 13b are scanned images of the optical textures of optical cells taken through a polarized light microscope and prepared with 5CB. The glass slides used to prepare the optical cells contained a surface with obliquely deposited gold on it which was immersed in an ethanolic solution of 1 mM hexanethiol for 90 minutes at 25° C. FIG. 13a is a scanned image obtained from the slide prepared as described above after overnight immersion in an aqueous TE buffered solution and treatment with the liquid crystal. FIG. 13b is a scanned image of the slide prepared as above after overnight immersion in an aqueous TE buffered solution containing 5′-GAT-CAG-CCA-CCG-GAA-CTG-CA-3′ at a concentration of 1 μM and a temperature of 25° C. A comparison of FIG. 13a with FIG. 13b shows that there is no apparent change in the ability of the surface to anchor liquid crystal. This provides evidence that no adsorption of the DNA fragment has occurred. This was expected because no thiol group was present on the DNA fragment and no complementary strand or fragment of DNA was present on the surface. This provides evidence that non-specific adsorption of non-complementary DNA will not occur in the DNA hybridization surfaces of the present invention. The thicknesses of the DNA and alkanethiol on the metallized surface for each of the slides used were 11.2 Å (1.12 nm) (FIG. 13a) and 11.5 Å (1.15 nm) (FIG. 13b) as measured using ellipsometry. The absence of thickness increase after incubation in the solution containing the nonthiolated DNA fragment is further evidence that non-specific adsorption of DNA is not a problem with the DNA hybridization surface of the present invention.

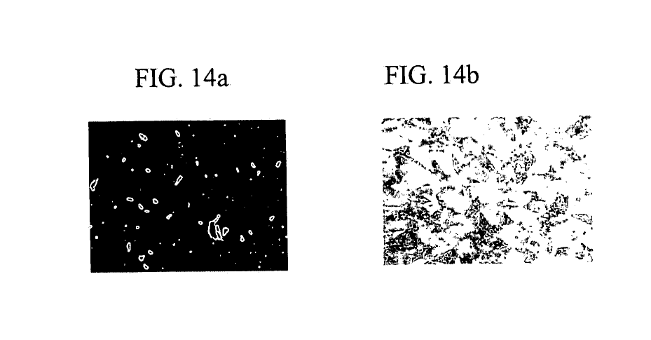

FIGS. 14a and 14b are scanned images of the optical textures of optical cells taken through a polarized light microscope and prepared with 5CB. The glass slides used to prepare the optical cells contained a surface with obliquely deposited gold on it which was immersed in an ethanolic solution of 1 mM hexanethiol for 60 minutes at 37° C. and then for 30 minutes at 25° C. in an aqueous KH₂PO₄ buffered solution containing 5′-HS—(CH₂)₆-TGC-AGT-TCC-GGT-GGC-TGA-TC-3′ at a concentration of 0.5 μM. FIG. 14a is a scanned image obtained from the slide prepared as described, and FIG. 14b is a scanned image of the slide prepared as above after incubation for 3 hours in an aqueous TE buffered solution containing 5′-GAT-CAG-CCA-CCG-GAA-CTG-CA-3′ at a concentration of 1 mM and a temperature of 25° C. The significant and readily apparent increase in non-uniformity of the liquid crystal that occurred upon incubation in a solution with a complementary target DNA fragment indicates that binding of the complementary DNA fragment disrupts the ability of the DNA hybridization surface to uniformly anchor liquid crystals.

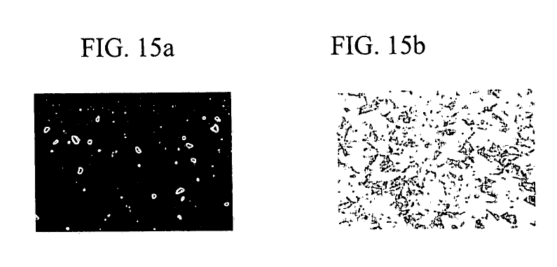

FIGS. 15a and 15b are scanned images of the optical textures of optical cells taken through a polarized light microscope and prepared with 5CB. The glass slides used to prepare the optical cells contained a surface with obliquely deposited gold on it which was immersed in an ethanolic solution of 1 mM hexanethiol for 60 minutes at 37° C. and then for 30 minutes at 25° C. in an aqueous KH₂PO₄ buffered solution containing 5′-HS—(CH₂)₆-TGC-AGT-TCC-GGT-GGC-TGA-TC-3′ at a concentration of 0.5 μM. FIG. 15a is a scanned image obtained from the slide prepared as described above, and FIG. 15b is a scanned image of the slide prepared as above after incubation for 24 hours in an aqueous TE buffered solution containing 5′-GAT-CAG-CCA-CCG-GAA-CTG-CA-3′ at a concentration of 1 μM and a temperature of 25° C. The significant and readily apparent increase in non-uniformity of the liquid crystal that occurred upon incubation in a solution with a complementary target DNA fragment indicates that binding of the complementary DNA fragment disrupts the ability of the DNA hybridization surface to uniformly anchor liquid crystals.

A comparison of FIGS. 14a, 14b, 15a, and 15b shows that DNA hybridization surfaces of the present invention may be used to detect complementary fragments of DNA in solutions of varying concentration.

It is understood that the invention is not limited to the embodiments set forth herein for illustration, but embraces all such forms thereof as come within the scope of the following claims.

What is claimed is:

1. A method for preparing a rinsed DNA hybridization surface, comprising:
rinsing a DNA hybridization surface with at least one rinsing solution to produce a rinsed DNA hybridization surface, prior to detecting the presence of a possible complementary DNA fragment or complementary strand of nucleic acids in a sample,
wherein the DNA hybridization surface comprises a support comprising a self assembled monolayer adsorbed on a metallized surface, wherein the self assembled monolayer comprises an alkanethiol and a strand of nucleic acids comprising a functional group that binds to the metallized surface of the support.

2. The method of claim 1, further comprising:
contacting the metallized surface of the support with the alkanethiol and the strand of nucleic acids that comprises the functional group to form the self assembled monolayer,
wherein the alkanethiol and the strand of nucleic acids comprising the functional group that binds to the metallized surface of the support are in one solution and are contacted with the metallized surface of the support at the same time.

3. The method of claim 1, further comprising:
contacting the metallized surface of the support with a first solution comprising the alkanethiol; and
contacting the metallized surface of the support with a second solution comprising the strand of nucleic acids that comprises the functional group;
wherein the first solution is contacted with the metallized surface of the support and then the second solution is contacted with the metallized surface of the support.

4. The method of claim 3, wherein the second solution is a phosphate buffered aqueous solution and the strand of nucleic acids that comprises the functional group is at a concentration ranging from 0.01 μM to 10 mM.

5. The method of claim 1, further comprising:
contacting the metallized surface of the support with a first solution comprising the alkanethiol; and
contacting the metallized surface of the support with a second solution comprising the strand of nucleic acids that comprises the functional group;
wherein the second solution is contacted with the metallized surface of the support and then the first solution is contacted with the metallized surface of the support.

6. The method of claim 5, wherein the second solution is a phosphate buffered aqueous solution and the strand of nucleic acids that comprises the functional group is at a concentration ranging from 0.01 μM to 10 mM.

7. The method of claim 1, wherein the metallized surface of the support comprises a top layer of gold.

8. The method of claim 7, wherein the top layer of gold has a thickness ranging from 5 nm to 30 nm.

9. The method of claim 7, wherein the top layer of gold overlies a layer of a material that promotes adhesion of the gold.

10. The method of claim 9, wherein the material that promotes adhesion of the gold is titanium.

11. The method of claim 9, wherein the layer of the material that promotes adhesion of the gold is a layer of titanium with a thickness ranging from 0.5 nm to 10 nm.

12. The method of claim 1, wherein the DNA hybridization surface is rinsed with at least two rinsing solutions.

13. The method of claim 12, wherein one of the at least two rinsing solutions is a phosphate buffered aqueous solution and at least one of the two rinsing solutions is water, an alcohol, or a combination of water and an alcohol.

14. The method of claim 13, wherein the DNA hybridization surface is first rinsed with the phosphate buffered aqueous solution and is then rinsed with the water, the alcohol, or the combination of water and the alcohol.

15. The method of claim 1, wherein the functional group of the strand of nucleic acids that binds to the metallized surface is a thiol group.

16. The method of claim 15, wherein the strand of nucleic acids comprising the thiol group comprises from 5 to 200 nucleic acids.

17. The method of claim 15, wherein the strand of nucleic acids comprising the thiol group comprises from 10 to 40 nucleic acids.

18. The method of claim 1, wherein the alkanethiol comprises from 4 to 20 carbon atoms.

19. The rinsed DNA hybridization surface produced according to the method of claim 1.
